SEQUENCE LISTING (61 sequences)

SEQ ID NO: 1 - protein, 5 aa, Artificial Sequence
  partial structure of cyclic peptide
  misc_feature (1)..(1): Xaa is Ile, Val or Leu, or N-Alkyl amino acid thereof
  misc_feature (2)..(2): Xaa is Ser or Thr, or N-Alkyl amino acid thereof
  misc_feature (3)..(3): Xaa is Lys or N-Alkyl amino acid thereof
  misc_feature (4)..(4): Xaa is Trp or N-Alkyl amino acid thereof
  misc_feature (5)..(5): Xaa is Trp, Tyr, His or Lys, or N-Alkyl amino acid thereof
XXXXX

SEQ ID NO: 2 - protein, 10 aa, Artificial Sequence
  partial structure of cyclic peptide
PLSKWWYSKR

SEQ ID NO: 3 - DNA, 2172 bp, Homo sapiens
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 4 - protein, 723 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 5 - DNA, 2187 bp, Homo sapiens
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 6 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 7 - protein, 9 aa, Artificial Sequence
  partial structure of cyclic peptide isolated peptide library based on RaPID system
SRKISKWYK

SEQ ID NO: 8 - protein, 9 aa, Artificial Sequence
  partial structure of cyclic peptide isolated peptide library based on RaPID system, HiP-1
TRKISKWYK

SEQ ID NO: 9 - protein, 9 aa, Artificial Sequence
  partial structure of cyclic peptide isolated peptide library based on RaPID system
ARKISKWWK

SEQ ID NO: 10 - protein, 9 aa, Artificial Sequence
  partial structure of cyclic peptide isolated peptide library based on RaPID system, HiP-12
KFRISKWYK

SEQ ID NO: 11 - protein, 9 aa, Artificial Sequence
  partial structure of cyclic peptide isolated peptide library based on RaPID system
SYKISKWYK

SEQ ID NO: 12 - protein, 9 aa, Artificial Sequence
  partial structure of cyclic peptide isolated peptide library based on RaPID system
VYKISKWYK

SEQ ID NO: 13 - protein, 9 aa, Artificial Sequence
  partial structure of cyclic peptide isolated peptide library based on RaPID system, HiP-13
IFKISKWYK

SEQ ID NO: 14 - protein, 9 aa, Artificial Sequence
  partial structure of cyclic peptide isolated peptide library based on RaPID system
SFKISKWYK

SEQ ID NO: 15 - protein, 9 aa, Artificial Sequence
  partial structure of cyclic peptide isolated peptide library based on RaPID system, HiP-14
QYKVSKWYK

SEQ ID NO: 16 - protein, 9 aa, Artificial Sequence
  partial structure of cyclic peptide isolated peptide library based on RaPID system
SKKVSKWYK

SEQ ID NO: 17 - protein, 9 aa, Artificial Sequence
  partial structure of cyclic peptide isolated peptide library based on RaPID system
SIRISKWYK

SEQ ID NO: 18 - protein, 9 aa, Artificial Sequence
  partial structure of cyclic peptide isolated peptide library based on RaPID system
QYKVSKWWK

SEQ ID NO: 19 - protein, 9 aa, Artificial Sequence
  partial structure of cyclic peptide isolated peptide library based on RaPID system
KYKINKWYK

SEQ ID NO: 20 - protein, 9 aa, Artificial Sequence
  partial structure of cyclic peptide isolated peptide library based on RaPID system
SYRISKWFK

SEQ ID NO: 21 - protein, 8 aa, Artificial Sequence
  partial structure of cyclic peptide isolated peptide library based on RaPID system, HiP-11
DYITKWWK

SEQ ID NO: 22 - protein, 9 aa, Artificial Sequence
  partial structure of cyclic peptide isolated peptide library based on RaPID system
PCKINKHWK

SEQ ID NO: 23 - protein, 9 aa, Artificial Sequence
  partial structure of cyclic peptide isolated peptide library based on RaPID system, HiP-15
NRGINKYWK

SEQ ID NO: 24 - protein, 8 aa, Artificial Sequence
  partial structure of cyclic peptide isolated peptide library based on RaPID system
CKITKWWK

SEQ ID NO: 25 - protein, 8 aa, Artificial Sequence
  partial structure of cyclic peptide isolated peptide library based on RaPID system
SYITKWWK

SEQ ID NO: 26 - protein, 9 aa, Artificial Sequence
  partial structure of cyclic peptide isolated peptide library based on RaPID system
KYKVTKWWK

SEQ ID NO: 27 - protein, 11 aa, Artificial Sequence
  partial structure of cyclic peptide isolated peptide library based on RaPID system, HiP-16
NKKVSKWYKRG

SEQ ID NO: 28 - protein, 8 aa, Artificial Sequence
  partial structure of cyclic peptide isolated peptide library based on RaPID system
CKINKFSR

SEQ ID NO: 29 - protein, 8 aa, Artificial Sequence
  partial structure of cyclic peptide isolated peptide library based on RaPID system, HiP-9
CKVSKWSK

SEQ ID NO: 30 - protein, 13 aa, Artificial Sequence
  partial structure of cyclic peptide isolated peptide library based on RaPID system, HiP-10
  misc_feature (6)..(6): Cyclic peptide may form with SH of this Cys
RYIKLCKISKWSR

SEQ ID NO: 31 - protein, 10 aa, Artificial Sequence
  partial structure of cyclic peptide isolated peptide library based on RaPID system, HiP-8
PLSKWWYSKR

SEQ ID NO: 32 - protein, 9 aa, Artificial Sequence
  partial structure of cyclic peptide isolated peptide library based on RaPID system, HiP-5
NKKITKWTA

SEQ ID NO: 33 - protein, 11 aa, Artificial Sequence
  partial structure of cyclic peptide isolated peptide library based on RaPID system, HiP-7
NQRVSKWKKQP

SEQ ID NO: 34 - protein, 9 aa, Artificial Sequence
  partial structure of cyclic peptide isolated peptide library based on RaPID system, HiP-6
NHKISKWKK

SEQ ID NO: 35 - protein, 13 aa, Artificial Sequence
  partial structure of cyclic peptide isolated peptide library based on RaPID system, HiP-4
QKISKHYKTNNKS

SEQ ID NO: 36 - protein, 8 aa, Artificial Sequence
  partial structure of cyclic peptide isolated peptide library based on RaPID system
DLSRWSIR

SEQ ID NO: 37 - protein, 8 aa, Artificial Sequence
  partial structure of cyclic peptide isolated peptide library based on RaPID system
DIARWSIR

SEQ ID NO: 38 - protein, 8 aa, Artificial Sequence
  partial structure of cyclic peptide isolated peptide library based on RaPID system
DRSRWSLL

SEQ ID NO: 39 - protein, 8 aa, Artificial Sequence
  partial structure of cyclic peptide isolated peptide library based on RaPID system, HiP-3
DSSRWSLR

SEQ ID NO: 40 - protein, 8 aa, Artificial Sequence
  partial structure of cyclic peptide isolated peptide library based on RaPID system
DSIRWSLG

SEQ ID NO: 41 - protein, 8 aa, Artificial Sequence
  partial structure of cyclic peptide isolated peptide library based on RaPID system, HiP-2
DHSRWSLR

SEQ ID NO: 42 - protein, 8 aa, Artificial Sequence
  partial structure of cyclic peptide isolated peptide library based on RaPID system
DTIRWSLR

SEQ ID NO: 43 - protein, 8 aa, Artificial Sequence
  partial structure of cyclic peptide isolated peptide library based on RaPID system
DVYRISIR

SEQ ID NO: 44 - protein, 8 aa, Artificial Sequence
  partial structure of cyclic peptide isolated peptide library based on RaPID system
DNFRISLR

SEQ ID NO: 45 - protein, 8 aa, Artificial Sequence
  partial structure of cyclic peptide isolated peptide library based on RaPID system
  misc_feature (7)..(7): Cyclic peptide may form with SH of this Cys
WIPNWVCL

SEQ ID NO: 46 - protein, 7 aa, Artificial Sequence
  partial structure of cyclic peptide isolated peptide librarybased on RaPID system
IAWVINV

SEQ ID NO: 47 - protein, 8 aa, Artificial Sequence
  partial structure of cyclic peptide, HiP8-1
DYITKWWK

SEQ ID NO: 48 - protein, 11 aa, Artificial Sequence
  partial structure of cyclic peptide, comparative example 1
NQRMSKWKKQP

SEQ ID NO: 49 - protein, 14 aa, Artificial Sequence
  partial structure of cyclic peptide, comparative example 2
QKISKHYKTNNKSC

SEQ ID NO: 50 - protein, 9 aa, Artificial Sequence
  partial structure of cyclic peptide, comparative example 3
NKKITKWTA

SEQ ID NO: 51 - protein, 8 aa, Artificial Sequence
  partial structure of cyclic peptide, comparative example 4
DHSRWSLR

SEQ ID NO: 52 - protein, 8 aa, Artificial Sequence
  partial structure of cyclic peptide, comparative example 5
DTIRWSLL

SEQ ID NO: 53 - protein, 13 aa, Artificial Sequence
  partial structure of cyclic peptide, analogue 1
VLSKWHPKLSKEL

SEQ ID NO: 54 - protein, 14 aa, Artificial Sequence
  partial structure of cyclic peptide, analogue 2
KLSKWYKRPANWKS

SEQ ID NO: 55 - protein, 13 aa, Artificial Sequence
  partial structure of cyclic peptide, analogue 3
ELSKWHPKLSRVL

SEQ ID NO: 56 - protein, 14 aa, Artificial Sequence
  partial structure of cyclic peptide, analogue 4
FLSKWKVNKHYKIP

SEQ ID NO: 57 - protein, 10 aa, Artificial Sequence
  partial structure of cyclic peptide, analogue 5
PLSKWWSQSR

SEQ ID NO: 58 - protein, 10 aa, Artificial Sequence
  partial structure of cyclic peptide, analogue 6
PLSKWYKQAR

SEQ ID NO: 59 - protein, 10 aa, Artificial Sequence
  partial structure of cyclic peptide, analogue 7
PLSKWWSHNR

SEQ ID NO: 60 - protein, 10 aa, Artificial Sequence
  partial structure of cyclic peptide, analogue 8
PLSKWWHQKN

SEQ ID NO: 61 - protein, 10 aa, Artificial Sequence
  partial structure of cyclic peptide, analogue 9
PLSKWWFSKK

(19) United States
(12) Patent Application Publication
(10) Pub. No.: US 2021/0395310 A1
(43) Pub. Date: Dec. 23, 2021

(54) Cyclic Peptide

(71) Applicant: The University of Tokyo, Tokyo (JP)

(72) Inventors: Hiroaki Suga, Tokyo (JP); Toby Passioura, Tokyo (JP); Kenichiro Ito, Tokyo (JP); Kunio Matsumoto, Tokyo (JP); Katsuya Sakai, Tokyo (JP); Hiroki Sato, Tokyo (JP)

(21) Appl. No.: 17/272,444

(22) PCT Filed: Sep. 6, 2019
(86) PCT No.: PCT/JP2019/035277

§ 371 Date: Sep. 7, 2021

(30) Foreign Application Priority Data
Sep. 6, 2018 (JP) 2018-167102

(51) Int. Cl. C07K 7/56; A61K 47/60; A61P 35/00; A61K 51/08

(52) U.S. Cl. CPC C07K 7/56; A61K 47/60; A61K 38/00; A61K 51/08; A61P 35/00

(57) ABSTRACT

The purpose of the present invention is to provide a cyclic peptide having any unit structure selected from the structures represented by the following formula (1):

—X¹—X²—X³—X⁴—X⁵— (1)
(in the formula (1), X¹ is I, V or L, or an N-alkylamino acid thereof,
   X² is S or T, or an N-alkylamino acid thereof,
   X³ is K or an N-alkylamino acid thereof,
   X⁴ is W or an N-alkylamino acid thereof, and
   X⁵ is W, Y, or H, or K or an N-alkylamino acid thereof), or a pharmaceutically acceptable salt of the cyclic peptide.

### TECHNICAL FIELD

The present invention relates to a cyclic peptide, a hepatocyte growth factor inhibitor containing the cyclic peptide and the like.

### BACKGROUND ART

Activation of a receptor by a cell growth factor is engaged in the repair and/or regeneration of tissues and organs. On the other hand, it causes cell cancerization and/or malignant progression. A hepatocyte growth factor (which may also be called “HGF”) is a growth factor having MET as a receptor (for example, Non-Patent Documents 1 to 4).

MET is a transmembrane receptor and has a tyrosine kinase domain in the cytoplasm.

HGF is secreted from cells as a single-chain HGF (which may also be called “scHGF”) and this scHGF is an inactive factor which is incapable of activating MET. This scHGF is subjected to extracellular processing (cleavage) by a specific protease and is to be converted into two-chain HGF (which may also be called “tcHGF”) which is a factor capable of activating MET. In short, scHGF is inactive precursor HGF and tcHGF is active HGF.

Since activation of a MET receptor system by HGF (which may also be called “HGF-MET receptor system hereinafter) in various cancer cells is involved in cell cancerization and/or malignant progression such as cancerization, invasion and metastasis of cancer cells, development of resistance (tolerance) to anticancer agents including molecular targeted agents, development of resistance to therapies with radiation or the like, and maintenance, invasive growth and the like of cancer stem cells, molecules inhibiting the HGF-MET receptor system including molecules inhibiting the activity of HGF become a candidate for anticancer agents (for example, Non-Patent Documents 1 to 4).

A plurality of research groups has so far reported that HGF is mostly present as scHGF in normal tissues, while in cancer tissues, it is converted into tcHGF by the processing with protease on the cancer cell surface, so that tcHGF is localized in cancer cells (cancer microenvironment) (for example, Non-Patent Documents 5 and 6).

Gefitinib, one of anticancer agents, serves to inhibit an epidermal growth factor receptor (EGF receptor) and inhibits growth of cancer cells, but cancer cells acquire resistance to gefitinib as a result of continuous long-term administration. It is reported that this occurs because the long-term administration of gefitinib increases HGF, activates MET, and activates a PI3K Akt pathway independently of the EGF receptor (Non-Patent Document 7).

### CITATION LIST

### Non-Patent Documents

Non-Patent Document 1: De Silva D M, Roy A, Kato T, Cecchi F, Lee Y H, Matsumoto K, Bottaro D P. Targeting the hepatocyte growth factor/Met pathway in cancer. Biochem Soc Trans, 45: 855-870, 2017.
Non-Patent Document 2: Petrini I. Biology of MET: a double life between normal tissue repair and tumor progression. Ann Transl Med, 3: 82, 2015.
Non-Patent Document 3: Sakai K, Aoki S, Matsumoto K. Hepatocyte growth factor and Met in drug discovery. J Biochem, 157: 271-284, 2015.
Non-Patent Document 4: Gherardi E, Birchmeier W, Birchmeier C, Vande Woude G F. Targeting MET in cancer: Rationale and progress. Nat Rev Cancer, 12: 89-103, 2012.
Non-Patent Document 5: Kataoka H, Hamasuna R, Itoh H, Kitamura N, Koono M. Activation of hepatocyte growth factor/scatter factor in colorectal carcinoma.

Cancer Res, 60: 6148-6159, 2000.
Non-Patent Document 6: Kawaguchi M, Kataoka H. Mechanisms of hepatocyte growth factor activation in cancer tissues. Cancers, 6:1890-1904, 2014.
Non-Patent Document 7: Yano, Matsumoto, et al. HGF induces gefitinib resistance of lung adenocaricinoma with EGF Receptor activating mulations. Cancer Res., 68: 9479-9487, 2008.

### SUMMARY

### Technical Problem

Although clinical tests of an anti-HGF antibody, an anti-MET antibody, and the like have so far been carried out with an HGF-MET receptor system as a target, an effective method capable of inhibiting the HGF-MET receptor system has not yet been established.

Crizotinib, cabozantinib and the like are known as a MET-tyrosine kinase inhibitor but they have not yet been found as a clinically effective agent capable of inhibiting the HGF-MET receptor system.

Considering that as described above, existing anticancer agents such as gefitinib acquire resistance, increase HGF and promote survival and growth of cancer cells, there is a demand for an excellent hepatocyte growth factor inhibitor (HGF inhibitor).

With the above-described problem in view, the present invention has been made and an objective is to provide a novel cyclic peptide as an excellent HGF inhibitor.

### Solution to Problem

The present inventors have carried out an intensive investigation in order to overcome the above-described problem. As a result, it has been found that a cyclic peptide having a specific structure is capable of significantly inhibiting an HGF-MET receptor system, leading to the completion of the present invention.

The present invention is as follows:

[1] A cyclic peptide having any unit structure selected from structures represented by the formula (1):

—X¹—X²—X³—X⁴—X⁵— (1)

(in the formula (1),

X¹ is I, V, or L, or an N-alkylamino acid thereof,

X² is S or T, or an N-alkylamino acid thereof,

X³ is K or an N-alkylamino acid thereof,

X⁴ is W or an N-alkylamino acid thereof, and

X⁵ is W, Y, H, or K, or an N-alkylamino acid thereof), or a pharmaceutically acceptable salt of the cyclic peptide.

[2] The cyclic peptide or pharmaceutically acceptable salt thereof as described above in [1], wherein the X¹ is I or L, or an N-alkylamino acid thereof.

[3] The cyclic peptide or pharmaceutically acceptable salt thereof as described above in [1] or [2], wherein the number of amino acid residues constituting a cyclic structure is from 8 to 17.

[4] The cyclic peptide or pharmaceutically acceptable salt thereof as described above in any of [1] to [3], wherein the cyclic peptide has an N—CO—CH₂—S structure.

[5] The cyclic peptide or pharmaceutically acceptable salt thereof as described above in [4], wherein the N is derived from an amino group of tryptophan.

[6] The cyclic peptide or pharmaceutically acceptable salt thereof as described above in [4] or [5], wherein the S is derived from a thiol group of cysteine.

[7] A hepatocyte growth factor (HGF) inhibitor, containing the cyclic peptide or pharmaceutically acceptable salt thereof as described above in any of [1] to [6],

[8] A pharmaceutical composition, containing the HGF inhibitor as described above in [7],

[9] The pharmaceutical composition as described above in [8], for use in the treatment or prevention of cancer-related diseases.

[10] A method of detecting active MET and/or tcHGF, including using the cyclic peptide or pharmaceutically acceptable salt thereof as described above in any of [1] to [6],

[11] A method of imaging a cancer tissue, including using positron emission tomography (PET) or fluorescence detection or chemiluminescence detection, or a combination thereof with the cyclic peptide or pharmaceutically acceptable salt thereof as described above in any of [1] to [6],

[12] The cyclic peptide or pharmaceutically acceptable salt thereof as described above in any of [1] to [6], having a group for positron detection, a group for fluorescence detection and/or chemiluminescence detection, or a group for antibody staining detection.

[13] A detecting agent for active-MET and/or tcHGF, containing the cyclic peptide or pharmaceutically acceptable salt thereof as described above in [12],

[14] A cancer diagnostic agent, containing the cyclic peptide or pharmaceutically acceptable salt thereof as described above in [12],

[15] A PET contrast agent, containing the cyclic peptide or pharmaceutically acceptable salt thereof as described above in [12],

[16] A method of detecting active-MET and/or tcHGF in a subject, including:

bringing the cyclic peptide or pharmaceutically acceptable salt thereof as described above in [12] into contact with a tissue of the subject, followed by incubation; and

carrying out fluorescence detection, positron detection, or antibody staining detection.

[17] A positron emission tomography (PET) imaging method of a cancer tissue of a subject, including:

administering the cyclic peptide or pharmaceutically acceptable salt thereof as described above in [12] or the contrast agent as described above in [15] to the subject;

allowing the cyclic peptide, the pharmaceutically acceptable salt thereof, or the contrast agent to penetrate the cancer tissue of the subject, and

acquiring a PET image of CNS or the cancer tissue of the subject.

### Advantageous Effects of Invention

The present invention provides a cyclic peptide. The cyclic peptide of the present invention has high affinity for HGF. The cyclic peptide of the present invention therefore binds to HGF and thereby inhibits activation of MET which is a receptor of HGF. In other words, the cyclic peptide of the present invention significantly inhibits the HFG-MET receptor system. In addition, the cyclic peptide of the present invention selectively recognizes tcHGF and binds thereto without binding to scHGF. Such a unique mechanism of inhibiting HGF-MET receptor system with the cyclic peptide makes it possible to take an approach different from that of existing anticancer agents and treat cancer.

In addition, the cyclic peptide of the present invention is capable of selectively binding to a portion of a cancer tissue where tcHGF is locally present as a result of interaction between tcHGF and MET, so that it is a molecule useful as a diagnostic tool which can be used for cancer detection and/or imaging. Further, the cyclic peptide of the present invention used as a diagnostic tool can diagnose the activation state of the HGF-MET receptor system and can be applied as a companion diagnostic agent serving for judgment basis for the use of various molecular targeted agents that inhibit the HGF-MET receptor system.

### BRIEF DESCRIPTION OF DRAWINGS

FIG. 1 shows curves of the measurement results of HGF, biotinylated HGF and fluoresceinated HGF by MET activation assay using cells. In EHMES-1 cells, HGF activated MET at EC50=0.07 nM. The arrow in the graph indicates 0.22 nM HGF used for peptide assessment. The biotinylated HGF and fluoresceinated HGF showed activity equivalent to that of non-labeled HGF. The EC50 value was determined by using dose-response (variable gradient, 4 parameters) curve fitting of Prism 6.0d and plotting the logarithm of MET activation against an HGF concentration. Data thus obtained are expressed as mean±s.d. of three independent measurement results.

FIG. 2 shows a concentration response curve of HiP-8 to MET activation induced by HGF in EHMES-1 cells. Data thus obtained are expressed as mean±s.d. often independent measurement results.

FIG. 3 shows a sequence alignment to be bound to HGF obtained by RaPID selection (CLC Sequence Viewer 8, Qiagen) and the results of inhibitory activity against MET activation induced by HGF in EHMES-1 cells.

FIG. 4 shows the results of chemical synthesis of analogs with high frequency in RaPID selection and inhibition tests against MET activation induced by HGF in EHMES-1 cells. Data thus obtained are expressed as mean±s.d. of two independent measurement results. The analogs were picked up from the DW library. The amino acid indicated by a lowercase alphabet is an N-methylated amino acid. Data thus obtained are expressed as mean±s.d. of three independent measurement results.

FIG. 5 shows a graph of concentration (nM) of HiP-8-PEG5 and HiP-8-PEG11, which are HiP-8-modified peptides, - % inhibition. Data thus obtained are expressed as mean±s.d. of three independent measurement results.

FIG. 6 shows the measurement results of fluorescein HGF obtained by HGF-MET binding assay. Binding to MET-immobilized beads or control protein G beads was detected by a flow cytometer at varying fluorescein HGF concentrations. The binding affinity of fluorescein HGF for MET was determined by applying binding saturation (one place) curve fitting of Prism 6.0d and plotting binding-HGF concentrations, resulting in KD=0.07 nM. The arrow in the graph indicates 0.44 nM fluorescein HGF used for peptide assessment. MFI means mean fluorescence intensity.

FIG. 7 shows a concentration response curve of HiP-8 to binding between fluorescein HGF and MET-beads. Data thus obtained are expressed as mean±s.d. of two independent measurement results.

FIG. 8 shows a concentration response curve of HiP-8-PEG5 and HiP-8-PEG11 to binding between fluorescein HGF and MET-beads. Data thus obtained are expressed as mean±s.d. of two independent measurement results.

FIG. 9 shows that HiP-8 inhibited gefitinib resistance induced by HGF. Cells were cultured for 3 days in the presence of ±1 μM gefitinib, 220 μM HGF and HiP-8. The line graph shows cell viability after treatment only with HiP-8.

FIG. 10 shows western blot figures showing that HiP-8 inhibited phosphorylation of MET, Gab1, Akt, and Erk1/2 induced by 2 nM HGF in EHMES-1 cells. The concentrations of HiP-8 are 10 nM, 100 nM, 1,000 nM, and 10,000 nM in order of mention from the third column.

FIG. 11 shows that HiP-8 inhibited B16-F10 melanoma migration induced by HGF at a level equivalent to that of an anti-HGF antibody.

FIG. 12 shows a binding rate between immobilized HGF and HiP, detected by surface plasmon resonance (SPR). RU represents a resonance unit (resonance unit). Data are expressed as mean±s.d. of typical sensorgrams performed in two iterations by using a plurality of concentrations of HGF.

FIG. 13 includes schematic views of HGF and HGF fragments.

FIG. 14 shows binding results of HGF to HiP-8. HGF was bound to immobilized HiP-8-PEG11 at KD=0.9 nM. The binding rate was detected by surface plasmon resonance (SPR). RU represents a resonance unit (resonance unit). The upward arrow and the downward arrow mean start and end of HGF input, respectively. Data are expressed as mean±s.d. of typical sensorgrams performed in two iterations by using HGFs having a plurality of concentrations.

FIG. 15 shows binding results of HGF fragment ‘tcHGF(Xa)’ to HiP-8 and HGF fragment ‘scHGF(Xa)’ to HiP-8. The tcHGF(Xa) was bound to immobilized HiP-8-PEG11 at KD=2.5 nM. A very limited portion of the scHGF(Xa) bound to HiP-8-PEG11. A binding rate was detected by surface plasmon resonance (SPR). RU represents a resonance unit (resonance unit). The upward arrow and the downward arrow mean start and end of HGF input, respectively. Data are expressed as mean±s.d. of typical sensorgrams performed in two iterations by using HGFs having a plurality of concentrations.

FIG. 16 shows a binding rate for the interaction between immobilized HIP-8-PEG11 and HGF fragments. Binding kinetic was analyzed using Biacore 3000. The upward arrow and the downward arrow mean start and end of HGF fragment input, respectively. Data are expressed as mean±s.d. of typical sensorgrams performed in two iterations by using HGF having a plurality of concentrations. A HiP-8-PEG11-unimmobilized sensor chip was used as a control. RU means a resonance unit (resonance unit).

FIG. 17 shows a binding rate for the interaction between immobilized HIP-8-PEG11 and HGF fragments. Binding kinetic was analyzed using Biacore 3000. The upward arrow and the downward arrow mean start and end of HGF fragment input, respectively. Data are expressed as mean±s.d. of typical sensorgrams performed in two iterations by using HGF having a plurality of concentrations. A HiP-8-PEG11-unimmobilized sensor chip was used as a control. RU means a resonance unit (resonance unit).

FIG. 18 shows uncompetitive inhibition of HGF-MET interaction by HiP-8. The binding rate of HGF(a), NK4(b) and SP(c) to the immobilized MET external domain was analyzed using Biacore 3000 at varying concentrations of HiP-8. A MET-unimmobilized sensor chip was used as a control. RU means a resonance unit (resonance unit). The upward arrow and the downward arrow mean start and end of HGF protein (HGF, NK4, or SP) input, respectively.

FIG. 19 shows the results of a structural change of HGF induced by HiP-8. HiP-8 did not show trypsin-induced proteolysis of the SP chain of HGF. HGF (2.5 μM)±HiP-8 (10 μM) was subjected to digestion treatment with trypsin at room temperature for 30 minutes. The digestion treatment was analyzed on from 5 to 20% SDS-PAGE under reduction conditions, followed by staining with Coomassie Blue. A trypsin:FHGF molar ratio is 0:1, 0.004:1, 0.012:1, 0.037:1, 0.11:1, 0.33:1, and 1:1 in order of mention from the left column.

FIG. 20 shows inhibition of MET activation/tyrosine phosphorylation by HiP-8-PEG11 in the mouse cancer tissue implanted with human lung cancer cells, (a) shows a time-dependent change of MET activation/tyrosine phosphorylation (pMET) after HiP-8 administration, (b) shows dose-dependent inhibition of MET activation/tyrosine phosphorylation.

FIG. 21 shows the results of performing contrast staining of the cell nucleus of the frozen human lung cancer tissue with 4′,6-diamidino-2-phenylindole (DAPI; product of Thermo Fisher Scientific) and analyzing the tissue section with a fluorescence microscope Biozero BZ-9000 (KEYENCE).

FIG. 22 shows the graph of intensities obtained by quantitatively determining the intensity of stained region and mixed region in respective tissues by ImageJ software (product of NIH) in order to semi-quantitatively detect antibody staining.

### DESCRIPTION OF EMBODIMENTS

Embodiments of the present invention will hereinafter be described in detail. It is to be noted that the present invention is not limited to the following embodiments but can be enforced in variously modified forms without departing from the gist of the invention.

The present invention relates to a cyclic peptide having any unit structure selected from the structures represented by the following formula (1):

—X¹—X²—X³—X⁴—X⁵— (1)

(in the formula (1), X¹ is I, V or L, or an N-alkylamino acid thereof,

X² is S or T, or an N-alkylamino acid thereof,

X³ is K or an N-alkylamino acid thereof,

X⁴ is W or an N-alkylamino acid thereof, and

X⁵ is W, Y, H, or K, or an N-alkylamino acid thereof), or a pharmaceutically acceptable salt of the cyclic peptide.

The cyclic peptide of the present invention significantly inhibits an HGF-MET receptor system.

In particular, the cyclic peptide of the present invention does not recognize scHGF incapable of activating MET but selectively recognizes tcHGF which is a molecular species capable of activating MET. An HGF inhibitor capable of selectively inhibiting tcHGF has an advantage not found in conventional inhibitors. Since only tcHGF is capable of activating MET, an HGF inhibitor capable of binding to tcHGF and detecting it selectively serves as a diagnostic molecular tool for detecting the activation of MET.

The term “cyclic peptide” as used herein means that it has, in the molecule, at least a cyclic structure composed of 5 or more amino acids. In addition to the cyclic structure, the cyclic peptide may have, as its molecular structure, a chain structure in which amino acids are linked with peptide bond or a structure other than a peptide structure.

The term “cyclic structure” as used herein means, in a linear peptide, a closed ring structure formed in the molecule by bonding, directly or via a linker or the like, two amino acids separated from each other by two or more amino acid residues.

The term “two amino acids separated from each other by two or more amino acid residues” means that the two amino acids have at least two amino acid residues therebetween.

The closed ring structure in the cyclic structure is not particularly limited but it is formed by a covalent bond between two amino acids.

Examples of the covalent bond between two amino acids include disulfide bond, peptide bond, alkyl bond, alkenyl bond, ester bond, thioester bond, ether bond, thioether bond, phosphonate ether bond, azo bond, C—S—C bond, C—N—C bond, C═N—C bond, amide bond, lactam bridge, carbamoyl bond, urea bond, thiourea bond, amine bond and thioamide bond.

When two amino acids bond to each other at their main chain, the closed ring structure is formed by a peptide bond. The covalent bond between two amino acids may be formed by a bond between the respective side chains of the two amino acids, a bond between the side chain and the main chain of the two amino acids, or the like.

The cyclic structure is not limited to that formed by a bond between the N-terminal and C-terminal amino acids of a linear peptide, but it may be formed by a bond between a terminal amino acid and a non-terminal amino acid or a bond between non-terminal amino acids. When one of the amino acids bonded for the formation of the cyclic structure is a terminal amino acid and the other one is a non-terminal amino acid, the resulting cyclic peptide has a cyclic structure having a linear peptide attached thereto like a tail.

The amino acid that forms the cyclic structure may be, as well as a natural amino acid, an artificial amino acid mutant and/or a derivative thereof.

Examples include natural proteinogenic L-amino acids, unnatural amino acids and chemically synthesized compounds having such properties known in the art as characteristics of an amino acid.

The proteinogenic amino acids are, when represented by three-letter code known in the art, Arg, His, Lys, Asp, Glu, Ser, Thr, Asn, Gin, Cys, Gly, Pro, Ala, lie, Leu, Met, Phe, Trp, Tyr and Val. The proteinogenic amino acids are, when represented by one-letter code known in the art, R, H, K, D, E, S, T, N, Q, C, G, P, A, I, L, M, F, W, Y and V.

The term “non-proteinogenic amino acids” means natural or unnatural amino acids other than proteinogenic amino acids.

Examples of the unnatural amino acids include α,α-disubstituted amino acids (such as α-methylalanine), N-alkylamino acids, D-amino acids, β-amino acids and α-hydroxy acids, each having a main chain structure different from that of natural amino acids; amino acids (such as norleucine and homohistidine) having a side-chain structure different from that of natural amino acids; amino acids (such as “homo” amino acids, homophenylalanine and homohistidine) having extra methylene in the side chain thereof; and amino acids (such as cysteic acid) obtained by substituting a carboxylic acid functional group in the side chain by a sulfonic acid group. Specific examples of the unnatural amino acids include amino acids described in WO2015/030014.

The N-alkylamino acid in the present invention is preferably an N-alkyl-α-amino acid, that is, an amino acid having an alkyl group bonded to an α-amino group thereof.

In the formula (1), X¹ is I, V or L, which is a branched-chain amino acid, or an N-alkylamino acid thereof, preferably, I or L, or an N-alkylamino acid thereof.

The number of amino acid residues constituting the cyclic structure is not particularly limited insofar as it is 5 or more. It may be, for example, 6 or more, 7 or more, 8 or more, 9 or more, or 10 or more and may be 30 or less, 25 or less, 20 or less, 17 or less, or 15 or less.

The number of amino acids constituting the cyclic structure is usually 5 or more and 30 or less. Within a range of 5 or more and 30 or less, the number of amino acids constituting the cyclic structure may be set at 6 or more, 8 or more, or 10 or more and set at 30 or less, 25 or less, 20 or less, or 15 or less.

The number of amino acids constituting the cyclic structure may be set at 8 or more and 20 or less, 8 or more and 17 or less, 9 or more and 17 or less, or 10 or more and 15 or less. It may be set at 10 or more and 13 or less or 10 or more and 12 or less.

The number of amino acid constituting the cyclic structure is preferably 9 or more and 17 or less, more preferably 10 or more and 15 or less, still more preferably 10 or more and 12 or less, from the standpoint of enhancing the MET activation inhibiting effect.

In the present invention, the cyclic peptide may be modified, for example, by phosphorylation, methylation, acetylation, adenylylation, ADP ribosylation, glycosylation, polyethylene glycol addition or the like, or it may be fused with another peptide and/or protein. The cyclic peptide may be biotinylated via an appropriate linker.

In the present invention, the cyclic peptide may be a dimer having two cyclic structures in the molecule, in which two cyclic peptides each having one cyclic structure are bound to each other via a linker structure, or it may have an intramolecular lactam bridge structure obtained by forming a lactam structure in the molecule.

The linker structure that connects two cyclic peptides is not particularly limited and those having a known structure that connects two peptides in the peptide synthesis field may be used.

The intramolecular lactam bridge structure may be formed by a bond between side chains of amino acids constituting the cyclic peptide. For example, an amino group of a side chain of Lys bonds to a carboxyl group of a side chain of Asp or Glu to form a peptide bond and thereby form an intramolecular lactam structure. Such a cyclic peptide thus has, in the molecule, another cyclic structure as a bridge structure. Instead of Lys, for example, DAP, DAB or Orn may bond to Asp or Glu.

The cyclic peptide of the present invention is not particularly limited insofar as it has, as the unit structure of the formula (1), a structure capable of inhibiting the HGF-MET receptor system. It has preferably an amino acid sequence which can be represented by the following formula:

(Z¹)ₘ-A-(Z²)ₙ

In the above formula, A is the unit structure of the formula (1) and m and n are arbitrary integers.

In the above formula, Z¹ and Z² are each independently an arbitrary amino acid and at least one of m and n is preferably an integer of 1 or more.

When m and n are each an integer of 1 or more, Z¹ and Z² preferably form a cyclic structure and thereby form a cyclic peptide. The cyclic structure may be formed by a bond between N-terminal Z¹ and C-terminal Z², bond between N-terminal Z¹ and non-C-terminal Z², bond between non-N-terminal Z¹ and C-terminal Z², or bond between non-N-terminal Z¹ and non-C-terminal Z².

Alternatively, Z¹ and Z² may form a cyclic structure via a linker.

When at least one of m and n is an integer of 1 or more, X¹ or X² bonds to Z² supposing that m is 0, and X⁴ or X⁵ bonds to Z¹ supposing that n is 0.

In the above formula, m and n preferably satisfy m+n of 1 or more and m and n are preferably integers independently selected so that m+n become 1 or more and 25 or less, m+n is selected as needed within a range of 1 or more and 25 or less and within a range of the number of amino acids constituting the cyclic structure.

In the cyclic peptide, the cyclic structure may be formed of an N—CO—CH₂-S structure and this structure is preferably formed by bond between an N-terminal acetyl group having a leaving group X instead of H such as chloroacetyl group which bonds to the amino group of Z³ and a thiol group of C-terminal side cysteine Cys. In this case, the formula is preferably represented by XCH₂CO—(Z³)—(Z⁴)_{P}-A-(Z⁵)_{q}—Cys-(X′)ᵣ.

Z³ to Z⁵ and X′ are each independently an arbitrary amino acid and p+q is arbitrarily selected from the number of amino acids constituting the cyclic structure and preferably within a range of 0 or more and 24 or less.

Z³ is preferably Trp; X′ at the C terminal is preferably Gly or Ser; and although r is not particularly limited insofar as it is an integer of 0 or more, it may be 20 or less, may be 10 or less, and is preferably 0 or 1.

In this specification, when r is 0, the cyclic peptide does not have X′, while when r is an integer of 2 or more, (X′)ᵣ of the cyclic peptide corresponds to the chain structure having amino acids linked to each other by a peptide bond.

Here, the —CO—CH₂—X group bonded to the N-terminal Z³ and the thiol group of Cys bond to each other to form a cyclic structure with the N—CO—CH₂—S structure. When (Z³)—(Z⁴)ₚ-A-(Z⁵)_{q} contains Cys anywhere, the N—CO—CH₂—S structure may be formed with this Cys.

The following is a specific example of the cyclic peptide of the present invention.

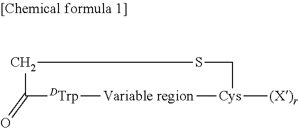

In the above structure, ^{D}Trp means the D form of Trp and S means the thiol group of Cys. In the drawing herein, the structure represented by —CH₂—CO— in the above structure is described also as Ac.

Variable region contains one or more structure(s) represented by the formula (1).

X′ is an arbitrary amino acid, and although r is not particularly limited insofar as it is an integer of 0 or more, it may be 20 or less, may be 10 or less, and is preferably 0 or 1.

Variable region is preferably represented by —(Z⁴)_{P}-A-(Z⁵)_{q}-A is the unit structure represented by the formula (1).

Z⁴ and Z⁵ are each independently an arbitrary amino acid, p+q is arbitrarily selected from the number of amino acids constituting the cyclic structure and preferably within a range of 0 or more and 24 or less, p+q is more preferably from 2 to 13, still more preferably from 5 to 10, still more preferably from 5 to 9.

p is preferably any integer selected from 1 to 3, more preferably an integer of 1 or 2, still more preferably 1.

q is preferably any integer selected from 2 to 10, more preferably any integer selected from 4 to 8, still more preferably 4.

Regarding p+q, it is preferable that p is any integer selected from 1 to 3 and at the same time, q is any integer selected from 2 to 10; it is more preferable that p is an integer of 1 or 2 and at the same time, q is any integer selected from 4 to 8; and it is still more preferable that p is 1 and at the same time q is 4.

One of preferred embodiments of the cyclic peptide of the present invention is represented by the following formula (I).

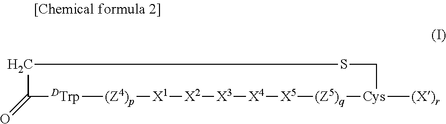

In the formula (I),

X¹ is I, V or L, or an N-alkylamino acid thereof,

X² is S or T, or an N-alkylamino acid thereof,

X³ is K or an N-alkylamino acid thereof,

X⁴ is W or an N-alkylamino acid thereof, and

X⁵ is W, Y, H or K, or an N-alkylamino acid thereof,

Z⁴ and Z⁵ are each independently an arbitrary amino acid,

p is any integer selected from 1 to 3,

q is any integer selected from 2 to 10,

X′ is an arbitrary amino acid, and

r is an integer of 0 or more.

p is any integer selected from 1 to 3, preferably an integer of 1 or 2, more preferably 1.

q is any integer selected from 2 to 10, preferably any integer selected from 4 to 8, more preferably 4.

Regarding p+q, p is any integer selected from 1 to 3 and at the same time, q is any integer selected from 2 to 10; it is preferable that p is an integer of 1 or 2 and at the same time, q is any integer selected from 4 to 8; and it is more preferable that p is 1 and at the same time q is 4.

When p in the formula (I) is 1, Z⁴ is preferably P, V, E, F, K or Y, or an N-alkylamino acid thereof, more preferably P or V, or an N-alkylamino acid thereof.

X¹ in the formula (I) is I, V or L, which is a branched amino acid, or an N-alkylamino acid thereof, preferably I or L, or an N-alkylamino acid thereof, more preferably I or an N-alkylamino acid thereof.

X² in the formula (I) is S or T, or an N-alkylamino acid thereof, preferably S or an N-alkylamino acid thereof.

X⁵ in the formula (I) is W, Y, H or K, or an N-alkylamino acid thereof, preferably W or an N-alkylamino acid thereof.

In the formula (I), —(Z⁵)_{q}— preferably contains at least one selected from Y, S, K or R, or an N-alkylamino acid thereof, more preferably contains at least two selected from Y, S, K or R, or an N-alkylamino acid thereof, more preferably contains —YSKR— (Y, S, K and R may each be an N-alkylamino acid).

Although r is not particularly limited insofar as it is an integer of 0 or more, it may be 20 or less, may be 10 or less, and is preferably 0 or 1.

Specific examples of the cyclic peptide represented by the formula (I) include cyclic peptides represented by the formula (1-1).

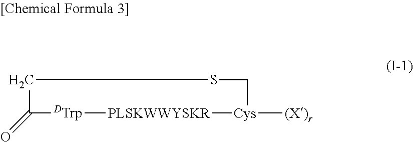

In the formula (1-1), X′ is an arbitrary amino acid and r is an integer of 0 or more.

In the formula (1-1), P, L, S, K, W, Y and R may each be an N-alkylamino acid.

Although r is not particularly limited insofar as it is an integer of 0 or more, it may be 20 or less, may be 10 or less, and is preferably 0 or 1.

The cyclic peptide of the present invention may be produced suitably by a translation synthesis method in a cell-free translation system.

It may be prepared by preparing a nucleic acid encoding the cyclic peptide and translating the nucleic acid in a cell-free translation system. The nucleic acid encoding the macrocyclic peptide may be designed as needed by those skilled in the art by using a genetic code used in an in vivo translation system, a reprogrammed genetic code, or a combination of them. The nucleic acid may be either DNA or RNA.

In accordance with the method using a cell-free translation system, an unnatural amino acid, similar to a natural amino acid, can be introduced efficiently into a peptide by using tRNA aminoacylated with the unnatural amino acid. For example, a tRNA having an arbitrary anticodon can be aminoacylated with an arbitrary natural or unnatural amino acid when an artificial aminoacyl tRNA synthetase flexizyme developed by the present inventors is used. By using this technology, therefore, it is possible to reprogram a genetic code composed of mRNA triplets so that it encodes amino acids different from those by a translation system in living body (WO2008/059823).

The cyclic peptide of the present invention can inhibit a hepatocyte growth factor (HGF). One aspect of the present inventions is therefore an HGF inhibitor containing the cyclic peptide or pharmaceutically acceptable salt thereof according to the present invention.

The HGF inhibitor of the present invention can be used as a pharmaceutical composition containing it. The pharmaceutical composition of the present invention can be used for the treatment or prevention of cancer-related diseases.

The administration route of the pharmaceutical composition is not particularly limited and it may be administered either orally or parenterally. Examples of the parenteral administration include administration by injection such as intramuscular, intravenous or subcutaneous injection, transdermal administration and transmucosal administration.

Examples of the administration route for transmucosal administration include nasal, ocular, pulmonary, vaginal and rectal ones.

The cyclic peptide in the pharmaceutical composition may be subjected to various modifications from the standpoint of pharmacokinetics such as metabolism and/or excretion. For example, the cyclic peptide can have longer residence time in blood or reduced antigenicity by adding thereto polyethylene glycol (PEG) or sugar chain.

The cyclic peptide may be contained in a biodegradable polymer compound such as polylactic acid-glycol (PLGA), porous hydroxyapatite, liposome, surface-modified liposome, emulsion prepared using an unsaturated fatty acid, nanoparticles, nanospheres or the like used as a sustained-release agent. When it is administered transdermally, it can be allowed to penetrate the stratum corneum by applying a weak electrical current through the skin surface (iontophoresis).

As the pharmaceutical composition, the cyclic peptide itself may be used as the effective ingredient, or it may be formulated by adding thereto a pharmaceutically acceptable additive or the like.

Examples of the dosage form include liquids and solutions (for example, injections), dispersions, suspensions, tablets, pills, powders, suppositories, powders, fine granules, granules, capsules, syrups, troches, inhalants, ointments, ophthalmic preparations, nasal preparations, ear preparations and cataplasms.

The pharmaceutical composition may be prepared in a conventional manner by using as needed an additive such as excipient, binder, disintegrant, lubricant, dissolving agent, solubilizing agent, colorant, taste/odor corrigent, stabilizer, emulsifier, absorption promoter, surfactant, pH regulator, antiseptic, wetting agent, dispersant or antioxidant.

Examples of the additive to be used for preparation of the composition include, but not particularly limited to, purified water, saline, phosphate buffer, dextrose, glycerol, pharmaceutically acceptable organic solvents such as ethanol, animal or vegetable oils, lactose, mannitol, glucose, sorbitol, crystalline cellulose, hydroxypropyl cellulose, starch, corn starch, silicic anhydride, magnesium aluminum silicate, collagen, polyvinyl alcohol, polyvinylpyrrolidone, carboxyvinyl polymer, carboxymethylcellulose sodium, sodium polyacrylate, sodium alginate, water-soluble dextran, carboxymethyl starch sodium, pectin, methyl cellulose, ethyl cellulose, xanthan gum, gum arabic, tragacanth, casein, agar, polyethylene glycol, diglycerin, glycerin, propylene glycol, petrolatum, paraffin, octyl dodecyl myristate, isopropyl myristate, higher alcohol, stearyl alcohol, stearic acid and human serum albumin.

As the absorption promoter in transmucosal absorption, surfactants such as polyoxyethylene lauryl ethers, sodium lauryl sulfate and saponin; salts of a bile acid such as glycocholic acid, deoxycholic acid and taurocholic acid; chelating agents such as EDTA and salicylic acids; fatty acids such as caproic acid, capric acid, lauric acid, oleic acid, linoleic acid and mixed micelle; enamine derivatives; N-acylcollagen peptide; N-acylamino acid; cyclodextrins; chitosans; and nitric oxide donors may be used.

Tablets and pills may also be coated with sugar, a material that dissolves in the stomach, a material that dissolves in the intestine or the like.

Liquids and solutions may contain distilled water for injection, physiological saline, propylene glycol, polyethylene glycol, a vegetable oil, an alcohol or the like. They may further contain a wetting agent, an emulsifier, a dispersant, a stabilizer, a dissolving agent, a solubilizing agent, an antiseptic or the like.

The present invention also provides a method of treating or preventing a disease of a patient by administering the HGF inhibitor to the patient in need thereof.

The dosage of the HGF inhibitor of the present invention may be determined as needed by those skilled in the art, depending on the symptom, age, sex, weight and sensitivity difference of a patient in need thereof, an administration method, an administration interval, the type of the agent and the like.

The patient is a mammal, preferably a human.

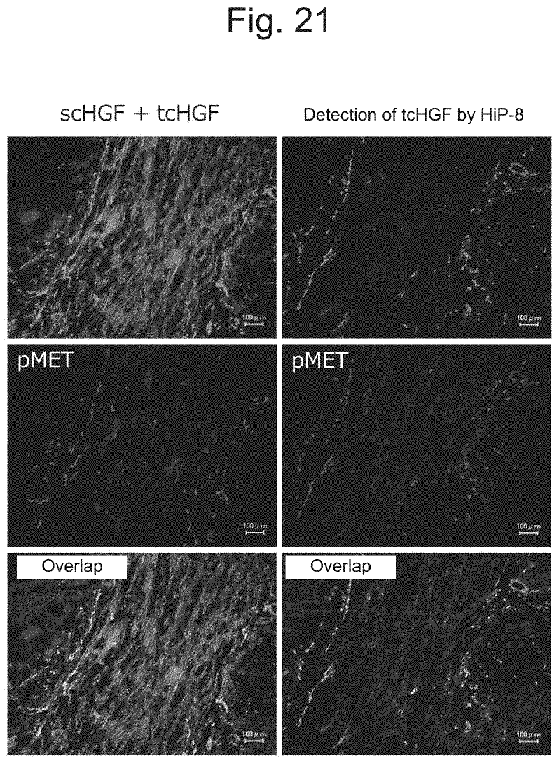

The cyclic peptide of the present invention is capable of selectively recognizing tcHGF and binding thereto. Since tcHGF and MET interact with each other, tcHGF is localized in the cancer tissue. Since the cyclic peptide of the present invention selectively binds to localized tcHGF, detecting the cyclic peptide bound to tcHGF enables detection and/or imaging of the cancer tissue. When one of the cyclic peptides of the present invention was actually used for staining of the cancer tissue, local presence of the cyclic peptide was observed in a tcHGF positive region as shown in FIG. 21. The cyclic peptide can be used for detection of tcHGF and also detection of activated MET. In addition, the cyclic peptide of the present invention is therefore useful as a detection and/or imaging tool of the activated state of MET in the cancer tissue and by using an already established versatile imaging method in combination, it can be used for detection/diagnosis of various diseases such as cancer and/or imaging of biotissue. Preferred examples of an imaging method of the cancer tissue include a positron emission tomography (PET) imaging method. Additional examples of the cancer tissue imaging method include fluorescence detection and chemiluminescence detection, and detection using them in combination.

Thus, the cyclic peptide of the present invention can be used for the detection of active MET and/or tcHGF.

For the detection of active MET and/or tcHGF with the cyclic peptide of the present invention, the cyclic peptide preferably has a group for positron detection, a group for fluorescence detection and/or chemiluminescence detection, or a group for antibody staining detection. In the present specification, the cyclic peptide of the present invention having a group for positron detection, fluorescence detection, chemiluminescence detection, or antibody staining detection may also be called “detection group-modified cyclic peptide”.

Preferred examples of the group for fluorescence detection include organic groups showing fluorescent properties. Preferably, the organic group showing fluorescent properties can be excited sufficiently in a spectrum region from 350 nm to 200 nm and has a useful emission zone in a spectrum region from 350 nm to 950 nm.

The group for fluorescence detection may be introduced into the cyclic peptide of the present invention by modifying the cyclic peptide of the present invention with a known fluorescent material. The introduction of the group for fluorescence detection may be performed in advance before the cyclic peptide is brought into contact with a subject or may be performed after the contact with the subject (in other words, when the peptide and tcHGF are interacting with each other).

Examples of the fluorescent material include eosin dyes, Toluidine Blue O, Methylene Blue, DAPI, Acridine Orange, DRAQ5, Hoechst 33342 and 33528, Calcein-AM, propidium iodide, Nile Blue, Nile Red, Oil Red O, Congo Red, Fast Green FCF, DiI, DiO, Did, TOTO (registered trademark), YO-PRO (registered trademark), Neutral red, New clear fast red, Pyronin Y, acid fuchsin, astrazone-family dyes, MitoTracker and other mitochondria dyes, LysoTracker and other lysosome dyes, safranine dyes, thioflavine dyes, fluorescent phalloidin, and plasma membrane dyes.

Preferred examples of the group for chemiluminescence detection include those emitting light when a partial structure thereof changes.

The group for chemiluminescence detection may be introduced into the cyclic peptide of the present invention by modifying the cyclic peptide of the present invention with a known chemiluminescent material. The introduction of the group for chemiluminescence detection may be performed in advance before the cyclic peptide is brought into contact with a subject or may be performed after the contact with the subject (in other words, when the peptide and tcHGF are interacting with each other).

Examples of the chemiluminescent material include dioxetane-based compounds such as AMPPD (registered trademark), CSPD (registered trademark), and CDP-Star (trademark). The dioxetane-based compounds are each hydrolyzed with an alkaline phosphatase, form an intermediate, and emit light as the intermediate is cleaved to produce adamantane.

Examples of the group for positron detection include groups containing a radioisotope atom selected from the group consisting of ²H, ³H, ¹¹C, ¹³N, ¹⁵O, ¹⁸F, from ⁷⁵Br to ⁷⁷Br, from ¹²³I to ¹³¹I, ⁶⁸Ga, ⁶⁴Cu, and ^{99m}Tc.

The group for positron detection may be introduced by synthesizing a cyclic peptide with an amino acid containing the radioisotope atom or may be introduced by modifying the cyclic peptide of the present invention with a known material containing the radioisotope atom. The introduction of the group for positron detection may be performed in advance before the cyclic peptide is brought into contact with a subject or may be performed after the contact with the subject (in other words, when the peptide and tcHGF are interacting with each other).

The introduction of the radioisotope atom into the cyclic peptide of the present invention may be achieved by referring to the method (method of labeling a peptide with radioactive iodine) described in Japanese Patent Application Laid-Open No. 2016-28096 and Japanese Patent Application Laid-Open No. 2016-047824, the method (an organotin reagent supported in an ionic liquid for the production of a radioactive pharmaceutical compound) described in Japanese Translation of PCT International Application Publication No. 2017-504658, and known methods relating to various complexes usable for the introduction of a radioisotope atom described in, for example, Jackson I M, Scott P J H, Thompson S. Clinical Applications of Radiolabeled Peptides for PET. Semin. Nucl. Med., 47: 493-523, 2017; Zeng D, Ouyang Q, Cai Z, Xie X Q, Anderson C J. New cross-bridged cyclam derivative CB-TE1K1P, an improved bifunctional chelator for copper radionuclides. Chem. Commun., 50: 43-45, 2014; Ding X, Xie H, Kang Y J. The significance of copper chelators in clinical and experimental application. J. Nutr. Biochem., 22: 301-310, 2011; and Pandya D N, Bhatt N, An G I, Ha Y S, Soni N, Lee H, Lee Y J, Kim J Y, Lee W, Ahn H, Yoo J. Propylene cross-bridged macrocyclic bifunctional chelator: a new design for facile bioconjugation and robust ⁶⁴Cu complex stability. J. Med. Chem., 57: 7234-7243, 2014.

In antibody staining, a fluorescence-labeled antibody specifically binds to an antigen and thereby a target molecule is fluorescence-labeled. The group for antibody staining detection is not particularly limited insofar as it is a group becoming an antigen of a fluorescence-labeled antibody. As the fluorescence-labeled antibody, a commercially available fluorescence antibody may be used, or an antibody labeled with the above-described fluorescence material may also be used.

Preferred examples of the fluorescence-labeled antibody include fluorescence-labeled avidin, and at that time, a group for antibody staining detection is preferably biotin.

An HRP-labeled antibody used as a primary antibody or secondary antibody may also be used preferably for chemiluminescence detection imaging.

The group for fluorescence detection, positron detection or antibody staining detection may bond to the cyclic peptide of the present invention directly via the functional group thereof such as amino group, carboxylic acid group, hydroxy group or thiol group, or may bond to it via a linker such as polyethylene glycol unit.

The detection group-modified cyclic peptide of the present invention can be used for the detection of active MET and/or tcHGF. The detection group-modified cyclic peptide of the present invention can therefore be used for the diagnosis of cancer in which active MET is expressed.

The detection group-modified cyclic peptide of the present invention, when it has a group for positron detection, can be used for PET contrast radiography.

The present invention therefore provides a detecting agent for active MET and/or tcHGF, cancer diagnostic agent, and PET contrast agent, each containing the detection group-modified cyclic peptide of the present invention.

The present invention also provides a method of detecting active MET and/or tcHGF in a subject.

The present detecting method includes bringing the detection group-modified cyclic peptide of the present invention or a pharmaceutically acceptable salt thereof into contact with the tissue of the subject, followed by incubation; and performing fluorescence detection, positron detection or antibody staining detection.

The present invention further provides a positron emission tomography (PET) imaging method of a cancer tissue of a subject.

The present imaging method includes administering the detection group-modified cyclic peptide or pharmaceutically acceptable salt thereof according to the present invention to the subject; allowing the cyclic peptide or pharmaceutically acceptable salt thereof to penetrate the cancer tissue of the subject; and acquiring a PET image of CNS or cancer tissue of the subject.

### EXAMPLES

HGF and domain protein of HGF molecule to be used in Examples were prepared as follows.

(Preparation of HGF Recombinant Protein)

Full-length human HGF cDNA (NM_001010932.2, isoform 3 hHGF del5 (DNA/AA), SEQ ID NO: 3; cDNA sequence, SEQ ID NO: 4; amino acid sequence) was used for the construction of all plasmids. Numbering of amino acid residues was based on the sequence of isoform 1 (NM_000601.5, SEQ ID NO: 5; cDNA sequence, SEQ ID NO: 6; amino acid sequence) containing further 5 amino acids in the K1 domain.

Regardless of the presence or absence of point mutations of elimination of N-linked glycosylation sites (N294Q, N402Q, T476G, N566Q and N653Q) or NK4 cDNA (residues Met1 to Val478), the full-length human HGF cDNA was cloned into pEHX1.1 plasmid. After HGF protein, HGFng (non-glycosylation) protein, and NK4 protein were expressed in Chinese hamster ovary (CHO) cells, secreted proteins were purified on a HiTrap heparin HP column (product of GE Healthcare), followed by a size exclusion chromatography on a Superdex 200 10/300 GL column (product of GE Healthcare) equilibrated with 20 mM Tris-HCl (pH 7.5) and 150 mM NaCl using AKTA purifier.

For the preparation of recombinant SP protein, the HGFng protein was digested with elastase (product of Sigma) (an HGF:elastase molar ratio=1:100) at 37° C. for 90 minutes. The reaction was terminated with 1 mM PMSF and then, the reaction mixture was loaded on a HiTrap Heparin HP column. A flow-through fraction containing SP was concentrated using an ultrafiltration membrane and then, the resulting fraction was subjected to size exclusion chromatography on a Superdex 200 10/300 GL column (product of GE Healthcare) equilibrated with 20 mM Tris-HCl (pH 7.5) and 150 mM NaCl.

The full-length HGF protein and N-terminally truncated HGF protein (Glu183-Ser728 for K2-4-SP and Gly388-Ser 728 for K4-SP) were mutated to substitute the cleavage site of wild-type HGF (KQLR/V) with the recognition sequence of Factor Xa (IEGR/V) in accordance with the previously reported method (Umitsu M, Sakai K, Ogasawara S, Kaneko M, Asaki R, Tamura-Kawakami K, Kato Y, Matsumoto K, Takagi J. Probing conformational and functional states of human hepatocyte growth factor by a panel of monoclonal antibodies. Scientific Rep, 6: 33149, 2016). Specifically, the above-described full-length HGF protein and N-terminally truncated HGF protein were obtained by adding a hexahistidine tag to the C terminal of the proteins, expressing them in Expi293F cells (product of Thermo Fisher Scientific), and purifying the secreted proteins on an Ni-NTA agarose column (Qiagen). The C-terminal His tag addition was achieved by overnight incubation with TEV protease. For the preparation of mature HGF (Xa), the TEV-treated sample was digested further with 6 μg/ml of Factor Xa (product of Novagen). The recombinant proHGF (Xa) and mature HGF (Xa) proteins were purified further on a Hitrap heparin HP column (GE Healthcare), followed by ion exclusion chromatography using a Superdex 75 10/300 GL column equilibrated with 20 mM tris-HCl (pH 7.5) and 150 mM NaCl. The N-terminally truncated recombinant HGF protein contained mutations of elimination of N-linked glycosylation sites (N294Q, N402Q, T476G, N566Q, and N653Q) or an unpaired cysteine (C561S). None of the mutations had an influence on the activity of HGF as previously reported (Fukuta K, Matsumoto K, Nakamura T. Multiple biological responses are induced by glycosylation-deficient hepatocyte growth factor. Biochem J, 388: 555-562, 2005).

[Preparation of Cyclic Peptide]

Cyclic peptides were obtained by the following procedure.

(Preparation of Cyclic Peptide Library)

A thioether peptide library was constructed with N-(2-chloroacetyl)-D-tryptophan (ClAcDW) as an initiator by using a flexible in vitro translation (FIT) system (Hipolito, C. J. & Suga, H. Ribosomal production and in vitro selection of natural product-like peptidomimetics: the FIT and RaPID systems. Curr Opin Chem Biol. 16, 196-203, 2012; and Passioura, T. & Suga, H. Flexizyme-mediated genetic reprogramming as a tool for noncanonical peptide synthesis and drug discovery. Chemistry. 19, 6530-6536, 2013).

An mRNA library corresponding to it was designed to have, in order of mention, a UGC codon encoding cysteine, from 4 to 15 NHK random codons (N means G, C, A or U, and K means G or U), and a stop codon of AUG (ClAcDW), and this random codons encoded proteinogenic amino residues. Theoretical diversity of the macrocycles based on the quantitative assessment of efficiencies of the individual transformation steps is at least 1012. After in vitro translation, a thioether bond was formed spontaneously between the N-terminal acetyl chloride group of the initiator DTrp residue and the sulfhydryl group of a downstream cysteine residue.

(Selection of Cyclic Peptide that Binds to hHGF)

Affinity selection was performed with a DW library against the full-length human HGF by employing the RaPID system (Hipolito, C. J. & Suga, H. Ribosomal production and in vitro selection of natural product-like peptidomimetics: the FIT and RaPID systems. Curr Opin Chem Biol. 16, 196-203, 2012; and Passioura, T. & Suga, H. Flexizyme-mediated genetic reprogramming as a tool for noncanonical peptide synthesis and drug discovery. Chemistry. 19, 6530-6536, 2013). The mRNA libraries and CIAc-^{D}Trp-tRNAfMetCAU were prepared as reported previously (Hipolito, C. J. & Suga, H. Ribosomal production and in vitro selection of natural product-like peptidomimetics: the FIT and RaPID systems. Curr Opin Chem Biol. 16, 196-203, 2012; Passioura, T. & Suga, H. Flexizyme-mediated genetic reprogramming as a tool for noncanonical peptide synthesis and drug discovery. Chemistry. 19, 6530-6536, 2013). Four μM mRNA library was ligated with 1.5 μM puromycin linker by using a T4RNA ligase at 25° C. for 30 minutes. The DNA of the puromycin linker bound to the 3′ terminal constant region of the mRNA libraries. After purification by phenol-chloroform extraction and ethanol precipitation, 1.4 μM mRNA-puromycin conjugate and 250 μm ClAc-D-Trp-tRNAfMetCAU were used in a methionine-deficient FIT system to generate respective peptide libraries. Next, an in vitro translation reaction was performed at 37° C. for 30 minutes, followed by incubation at 25° C. for 12 minutes to promote an mRNA-peptide composite. Then, incubation was performed at 37° C. for 30 minutes to facilitate peptide cyclization.

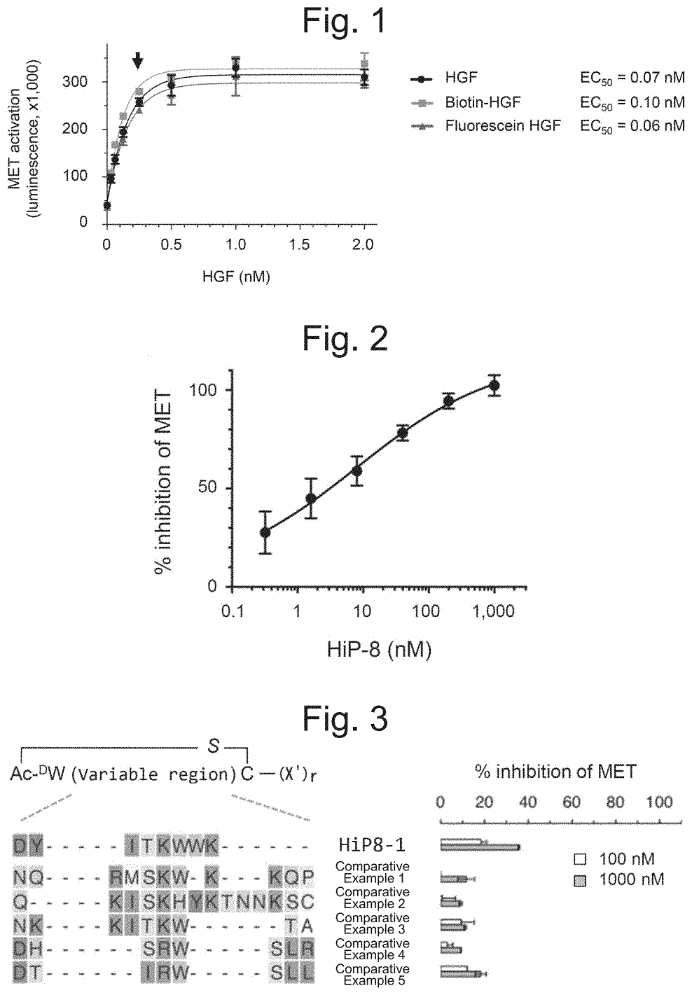

Then, the product was reverse-transcribed by RQ-RTase (Promega) at 42° C. for 1 hour to tag the cyclic peptide to the mRNA-cDNA hybrid. First, undesired bead binders were removed using a biotin-immobilized Dynabeads M-280 streptavidin (product of Life Technologies). This process is called “pre-cliarance” or “negative selection” and was repeated twice (six times from round 2). After the pre-clearance, the peptide-mRNA/cDNA solution was incubated for 30 minutes at 4° C. with 200 nM of the biotinylated full-length human HGF-immobilized Dynabeads M-280 streptavidin, and the hHGF binder was isolated. The biotinylation of hHGF was performed using a succinimidyl biotin labeling kit (Dojindo). It was found by the MET activation assay of EHMES-1 cells that bioactivity of the biotinylated hHGF was equivalent to that of non-labeled HGF (FIG. 1). This process is referred to as positive selection.

The fused peptide-mRNA/cDNA was isolated from the beads by incubating once in a PCR reaction buffer heated at 95° C. for 5 minutes. The amount of eluted cDNA was measured by quantitative PCR. The remaining cDNAs were amplified by PCR, purified, and transcribed into mRNA as a library for the next round of selection. The library preparation, pre-clearance and positive selection were one round of the enrichment processes. Beginning with round 2, the library was reversed-transcribed by M-MLV before the incubation with target protein. Significant enrichment of cDNAs was observed at the fourth round. The recovered cDNAs were ligated into pGEM-T-Easy vector (Promega) using TA-cloning. The vector was transformed into DH5a competent cells. Individual clones were picked, and their sequences were determined.

(Chemical Synthesis of Cyclic Peptide)

Cyclic peptides were synthesized using a Syro Wave Automated peptide synthesizer (Biotage) by Fmoc solid-phase peptide synthesis (SPPS) in accordance with the known method (Ito, K. et al. Artificial human Met agonists based on macrocycle scaffolds. Nature Communications 6, 6372, 2015).

Specifically, after a product was obtained by the automated synthesis, the chloroacetyl group for cyclization was coupled onto the N-terminal amide group. The peptide was cleaved with a solution of 92.5% trifluoroacetic acid (TFA), 2.5% water, 2.5% triisopropylsilane and 2.5% ethanedithiol, and precipitated by diethyl ether. To perform the cyclization reaction, the peptide pellets were dissolved in 10 ml of a 1:1 solution of water and DMSO/0.1% TFA and the resulting solution was adjusted to pH>8 by the addition of triethylamine and incubated for 1 hour at 42° C. TFA was added to the reaction solution to acidify the peptide suspension and the cyclization reaction was quenched. Then, the peptide was purified by reverse-phase HPLC (RP-HPLC) using Shimazu prominence LC-20AP system equipped with a Merck Chromolith Prep column (200-25 mm) and the molecular weight was verified by MALDI-TOF mass spectrometry (product of Bruker Daltonics) by using PerkinElmer Sciex API 150EX. As an alternative purification method, the cyclic peptide was purified using HyperSep SPE C18 column (product of Thermo Fisher Scientific).

The structures of human HGF-targeting cyclic peptides selected by RaPID system and chemically synthesized are shown below in Tables 1 to 3.

TABLE 1

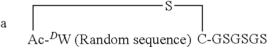

| Peptide | Sequence | Frequency |
|---|---|---|
| | SRKISKWYK | 1/43 |
| HiP-1 | TRKISKWYK | 3/43 |
| | ARKISKWWK | 1/43 |
| HiP-12 | KFRISKWYK | 1/43 |
| | SYKISKWYK | 1/43 |
| | VYKISKWYK | 1/43 |
| HiP-13 | IFKISKWYK | 1/43 |
| | SFKISKWYK | 1/43 |
| HiP-14 | QYKVSKVVYK | 1/43 |
| | SKKVSKWYK | 1/43 |
| | SIRISKWYK | 1/43 |
| | QYKVSKWWK | 1/43 |
| | KYKINKWYK | 1/43 |
| | SYRISKWFK | 1/43 |
| HiP-11 | DYITKVVWK | 1/43 |
| | PCKINKHWK | 1/43 |
| HiP-15 | NRGINKYWK | 1/43 |
| | CKITKWWK | 1/43 |
| | SYITKWWK | 1/43 |
| | KYKVTKWWK | 1/43 |
| HiP-16 | NKKVSKWYKRG | 1/43 |
| | CKINKFSR | 1/43 |
| HiP-9 | CKVSKWSK | 1/43 |
| HiP-10 | RYIKLC*KISKWSR | 1/43 |
| HiP-8 | PLSKWWYSKR | 1/43 |
| HiP-5 | NKKITKINTA | 1/43 |
| HiP-7 | NQRVSKWKKQP | 1/43 |
| HiP-6 | KHKISKWKK | 1/43 |
| HiP-4 | QKISKHYKTNNKS | 1/43 |

TABLE 2

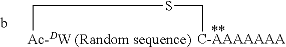

| Peptide | Sequence | Frequency |
|---|---|---|
| | DLSRWSIR | 1/43 |
| | DIARWSIR | 1/43 |
| | DRSRWSLL | 1/43 |
| HiP-3 | DSSRWSLR | 1/43 |
| | DSIRWSLG | 1/43 |
| HiP-2 | DHSRWSLR | 1/43 |
| | DTIRWSLR | 2/43 |
| | DVYRISIR | 1/43 |
| | DNFRISLR | 1/43 |

TABLE 3

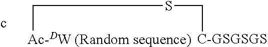

| Peptide | Sequence | Frequency |
|---|---|---|
| | WIPNWV*CL | 1/43 |
| | IAWVINV | 1/43 |

In the above tables, * shows that there is possibility of forming a thioether bond at cysteine *C contained in the Random sequence and ** shows occurrence of frame shift mutation.

[Assessment of Cyclic Peptide]

Various activity tests of the cyclic peptides thus obtained were performed.

The following are sources of cells used for activity assessment.

EHMES-1 cells were provided by Dr. Hamada (Ehime Univ., Japan).

B16-F10 and HCC827 cells were obtained from ATCC (Manassas, Va.).

FC-9 cells were obtained from Immuno-Biological Laboratories Co., Ltd (Gunma, Japan).

Unless otherwise specifically described, all the cell lines were cultured on a RPMI-1640 medium containing 10% fetal bovine serum (FBS) and 2 mM L-glutamine under the conditions of 37° C. and 5% CO₂.

(MET Activation Test)

EHMES-1 Cells were stimulated for 10 minutes while using a medium with 20 ng/ml (220 μM) of an hHGF protein in the presence or absence of a cyclic peptide and washed with phosphate-buffered physiological saline (PBS). Then, 4% paraformaldehyde (PFA) was added in PBS and the resulting mixture was incubated for 30 minutes, followed by further washing three times with PBS. The cells were fixed for 30 minutes with 5% goat serum and 0.02% Triton X-100 in PBS and then, incubated at 4° C. for 12 hours in Phospho-Met (Tyr1234/1235) XP rabbit mAb (D26, product of Cell Signaling Technology, Danvers, Commonwealth of Massachusetts), a monoclonal antibody. As the monoclonal antibody, used was that diluted at 1:1000 in a 1% goat serum PBS suspension. The cells were washed three times with PBS and incubated for 1 hour in a liquid obtained by diluting an HRP-conjugated anti-rabbit antibody and a 1% goat serum PBS suspension at 1:1000. After incubation, the cells were washed four times with PBS. By using a chemiluminescence method with ImmunoStar (registered trademark) LD reagent (product of Wako Pure Chemicals, Japan), chemiluminescence intensity was measured by ARVO MX (product of Perkin Elmer). Relative MET phosphorylation was calculated as the following: (chemiluminescence unit of sample—chemiluminescence unit of mock control)/(chemiluminescence unit of 220 μM HGF—chemiluminescence unit of mock control).

The results of the MET activation inhibition concentration of the cyclic peptide HiP-8 was 8 nM in terms of IC50. A HiP-8 concentration (nM)—inhibition (%) graph showing the results of the MET activation inhibition test of HiP-8 is shown in FIG. 2.

In the MET activation inhibition test of cyclic peptides HiP-11 (which may also be called “HiP8-1” hereinafter), HiP-1, Flip-6, and Flip-9 to 16, the cyclic peptides each had MET inhibitory activity at a concentration of 100 nM or more. The % inhibition of MET activation by the cyclic peptide HiP8-1 and comparative compounds are shown in FIG. 3.

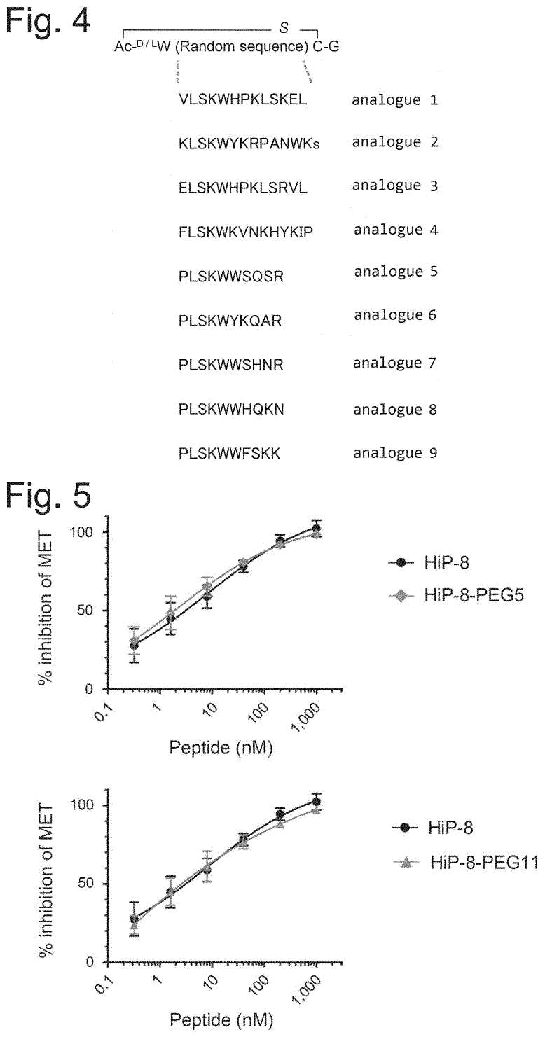

Further, the respective structures of the cyclic peptides having at least the structure represented by the formula (1) are shown in FIG. 4. The cyclic peptides shown in FIG. 4 were obtained in accordance with a method similar to that employed in the above [preparation of cyclic peptide]. The cyclic peptides shown in FIG. 4 had from 20 to 80% MET activation inhibition when they had a concentration of 100 nM or more.

FIG. 5 includes respective graphs of the concentrations of HiP-8-PEG5 and HiP-8-PEG11, which are HiP-8 modified peptides, and % inhibition. The HiP-8-PEG5 and HiP-8-PEG11 are represented by the following structure.

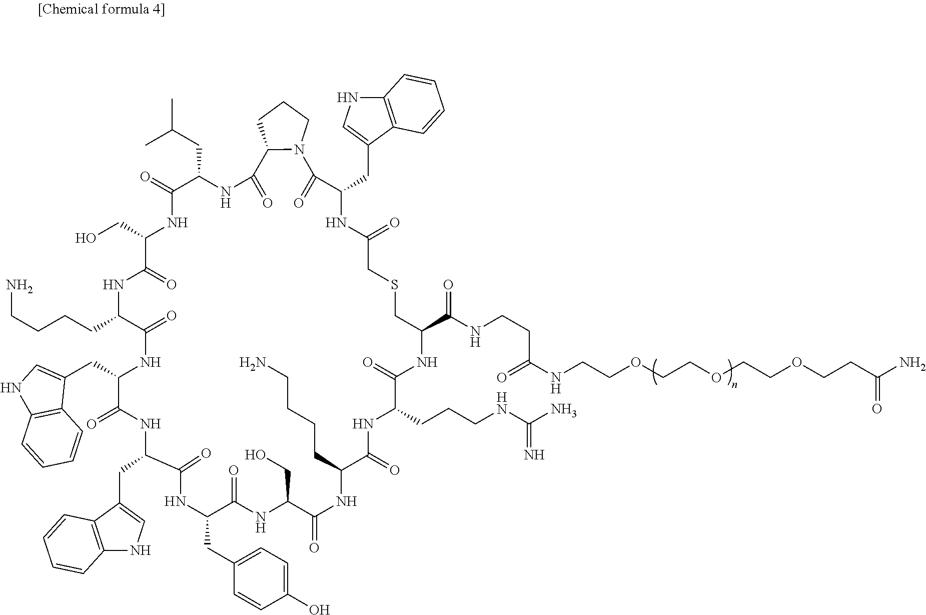

(In the structure, n is 4 or 9. The structure having n of 4 is HiP-8-PEG5 and the structure having n of 10 is HiP-8-PEG11).

(Binding Test of Fluorescein-HGF to MET-Fc Beads)

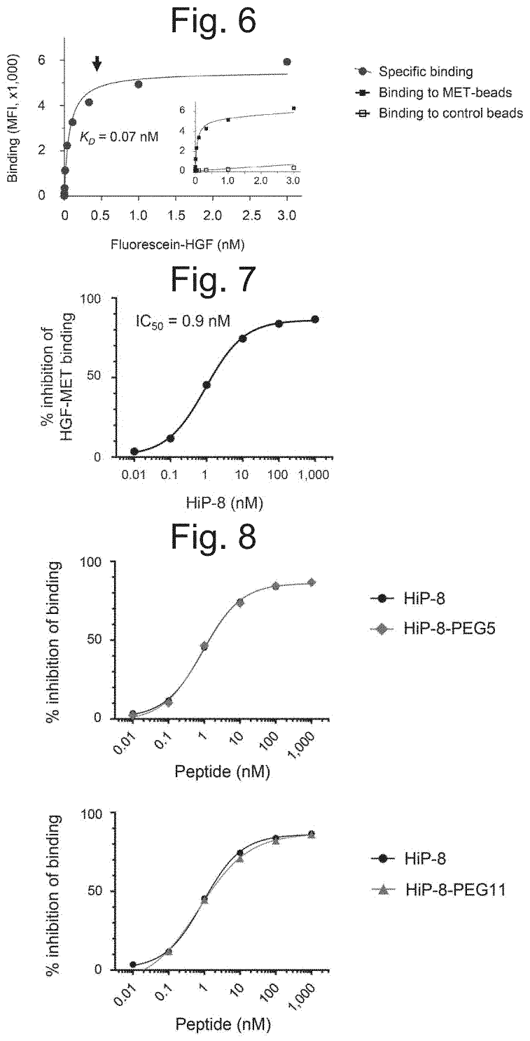

Labeling of hHGF with fluorescein was performed using a succinimidyl fluorescein labeling kit (product of Dojindo). On 200 μl (50% v/v) of protein G beads (Spherotech) in Tris-buffered physiological saline (pH 7.5) containing 0.1% BSA and 0.05 μl of Tween-20, 5 μg of MET-extracellular domain-Fc fusion protein (product of R&D systems) was immobilized. For each of gradually increased fluorescein-HGF concentrations, the protein was incubated at 25° C. for 1 hour together with 5 μl (50% v/v) protein G beads or MET-Fc-immobilized protein G beads in 200 μl of tris buffered physiological saline (pH 7.5) containing 0.1% BSA and 0.05% Tween. The fluorescence intensity of the beads was detected using a flow cytometer (product of FACS Canto II, BD Biosciences). The results of fluorescein-HGF binding affinity are shown in FIG. 6.

(Inhibition of HGF-MET Binding)

For a competitive test of the cyclic peptide, for each of increased concentrations of the cyclic peptide, 0.44 nM fluorescein-HGF and MET-Fc immobilized beads were mixed, the resulting mixture was incubated at 25° C. for 1 hour, and fluorescence intensity of the beads was measured using a flow cytometer.

The results of the competitive test of HiP-8 and fluorescein-HGF, that is, the results of the inhibition of HGF-MET binding are shown in FIG. 7. The results of the inhibition of HGF-MET binding by HiP-8-PEG5 and HiP-8-PEG11 are shown in FIG. 8.

With regards to the results of the MET activation test and the HGF-MET binding inhibition, a dose-response curve was made using Prism 6.0d (GraphPad) and curve fitting was performed. The IC50 value in the MET activation test and the HGF-MET binding inhibition was determined by plotting a % inhibition against a logarithmic compound concentration by using a dose-response (variable slope, four parameters) curve fitness function.

(Gefitinib Resistance Test)

The resistance to gefitinib, an anticancer agent, was tested by seeding human lung cancer cells, PC-9 cells, on a 24-well plate and cultured for 24 hours on a RPMI medium containing 10% fetal bovine serum. The number of living cells was counted after non-addition or addition of gefitinib (1 mM), HGF (20 ng/mL), or HiP8-PEG11 (1-1000 nM) and culturing for 72 hours.

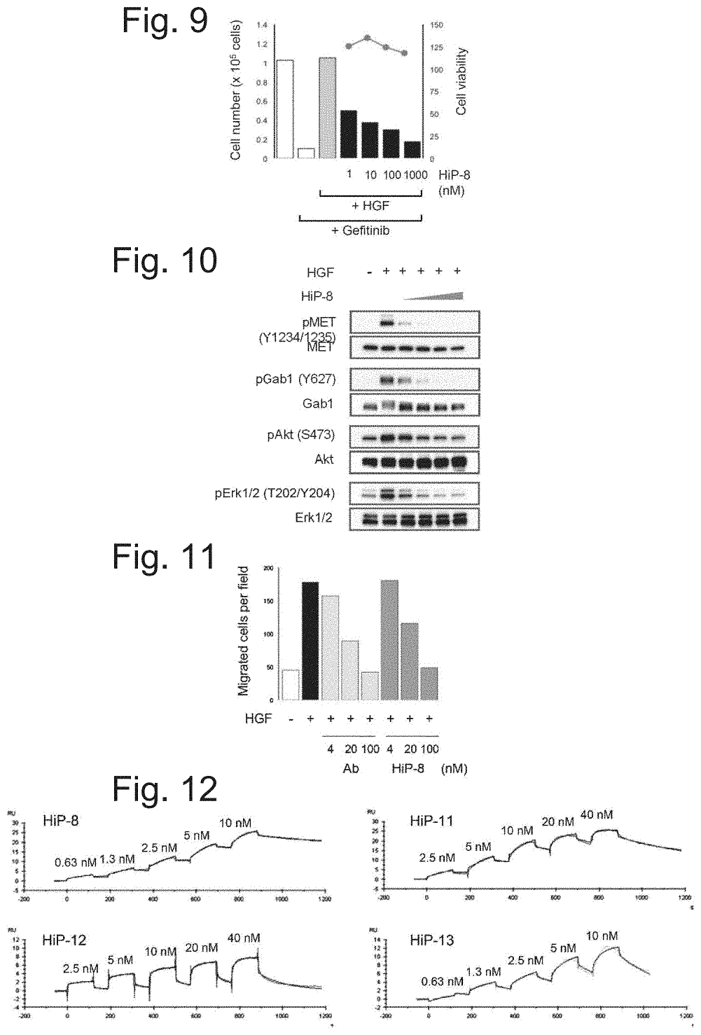

As shown in FIG. 9, gefitinib strongly inhibits growth of lung cancer cells at 1 mM concentration, while HGF suppresses the cell growth inhibitory action of gefitinib and produces resistance to gefitinib. HiP-8 inhibited gefitinib resistance induced by HGF.

(Western Blotting Analysis)

EHMES-1 cells were cultured on a 6-well plate until they were from 80 to 90% confluent. After starvation for 6 hours, the cells were stimulated for 10 minutes with 2 nM hHGF in a culture medium in the absence of a cyclic peptide or in the presence of 10, 100, 1,000, or 10,000 nM cyclic peptide. The resulting cells were washed with PBS and lysed with 200 μl of lysis buffer 17 (product of R&D Systems) containing 1× Complete protease inhibitor cocktail (product of Roche). The protein concentration was measured by BCA assay (product of Thermo Fisher Scientific), and the protein was provided for 10% polyacrylamide gel SDS-PAGE. The protein was transferred onto a PVDF (polyvinylidene difluoride) membrane (Bio-Rad) and detected using a Can Get Signal (registered trademark) solution 1 containing a primary antibody (diluted at 1:2000), that is, MET (CST, 25H2), phospho-MET (Tyr1234/1235) (CST, D26), Akt (CST, 11E7), phospho-Akt (Ser473) (CST, D9E), Erk1/2 (CST, 137F5), phospho-Erk1/2 (Thr202/Tyr204) (CST, D13.14.4E), Gab1 (CST) and phospho-Gab1 (Tyr627) (CST, C32H2), and Can Get Signal (registered trademark) solution 2 (product of Toyobo) containing a horseradish peroxidase-bound secondary antibody (product of Dako) (diluted at 1:5000). Chemiluminescence signals were developed using a Luminata Forte HRP substrate (product of Merck Millipore) and emission intensity was observed using ImageQuant LAS 350 (product of GE Healthcare).

Electrophoresis results are shown in FIG. 10.

(Cell Migration Test)

B16F10 cells (1×10⁵ cells/insert) cultured in 200 μl of a RPMI1640 medium containing 0.5% FBS were seeded in the upper insert (transwell having a diameter of 8 mm, product of Corning) while 800 μl of a RPMI1640 medium with or without hHGF or HiP-8, or an anti-human HGF rabbit polyclonal antibody and supplemented with 0.5% FBS was added to the bottom chamber. The cells were cultured for 16 hours and fixed with 4% PFA solution in PBS. The cells attached to the bottom surface of the membrane were stained with 0.4% crystal violet solution in 20% methanol and the number of cells was counted.

As shown in FIG. 11, HiP-8 suppressed cell migration at a level comparable to that of the anti-HGF antibody.

(Binding Test of Cyclic Peptide to HGF)

Binding of the cyclic peptides to immobilized HGF was measured using surface plasmon resonance (SPR) with Biacore T200 (product of GE Healthcare). Specifically, 2000 resonance units (RU) of biotinylated HGF was immobilized onto a CAP sensor chip by using a biotin capture kit (product of GE Healthcare).

Binding of the cyclic peptides was tested by injecting varying concentrations of the cyclic peptides into an HBS EP+buffer (10 mM HEPES (pH 7.4), 150 mM NaCl, 3 mM EDTA, and 0.05% (v/v) Surfactant P20) containing 0.1% DMSO at a flow rate of 30 μl/min. Binding of the cyclic peptides to HGF was analyzed by single cycle kinetics analysis.

The results are shown in FIG. 12. Time (second) is plotted along the abscissa and a resonance unit (RU) is plotted along the ordinate. An equilibrium dissociation constant K_{D} is shown in the following table. In the table, Kₐ represents an association rate and K_{d} represents a dissociation rate.

TABLE 4

| Peptide | Kₐ (10⁶ M⁻¹ s⁻¹) | K_{d} (10⁻³s⁻¹) | K_{D} (nM) |
|---|---|---|---|
| HiP-8 | 1.1 | 0.4 | 0.4 |
| HiP-11 | 1.3 | 1.6 | 1.3 |
| HiP-12 | 0.7 | 7.9 | 12 |
| HiP-13 | 1.9 | 4.0 | 2.1 |

Binding of HGF and HGF fragments to immobilized HiP8-PEG11 was measured by surface plasmon resonance (SPR) with Biacore 3000 (product of Ge Flealthcare). Hip8-PEG11-biotin was supported on the surface of a streptavidin-coated chip at 10 RU (resonance unit). The test was performed by injecting HGF and HGF fragments with varying concentrations into 10 mM TBS (pH 7.4), 150 mM NaCl, and 0.05% Tween 20 (v/v) at a flow rate of 30 μl/min. Binding affinity was analyzed from a fitting model of steady-state affinity by multi-cycle kinetics analysis.

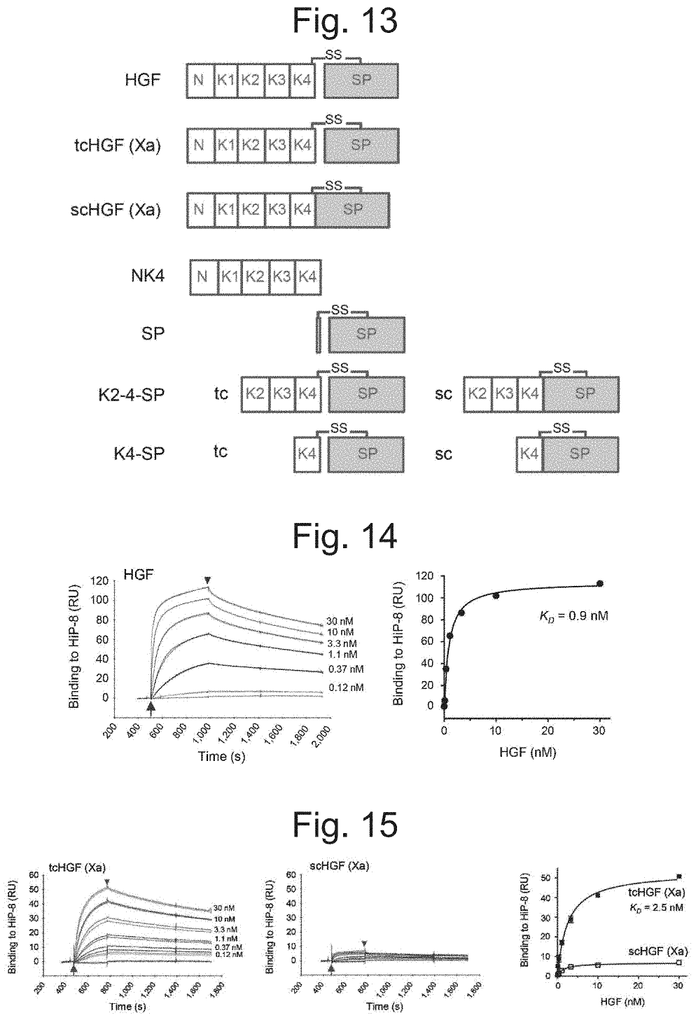

Schematic views of HGF and HGF fragments are shown in FIG. 13. HGF was an active type HGF without substitution of the cleavage site of wild type HGF (KQLR/V) with the recognition sequence of Factor Xa (IEGR/V) and domain-deficiency and was a two-chain HGF capable of activating MET. tcHGF (Xa) was an HGF with substitution of the cleavage site of the HGF with the recognition sequence of Factor Xa (IEGR/V) and was cleaved into two chains by the Factor Xa treatment. It was also a two-chain HGF capable of activating MET. On the other hand, scHGF (Xa) was obtained by substituting the cleavage site of HGF with the recognition sequence of Factor Xa (IEGR/V). It was a single-chain HGF not subjected to the Factor Xa treatment and was an inactive type incapable of activating MET.

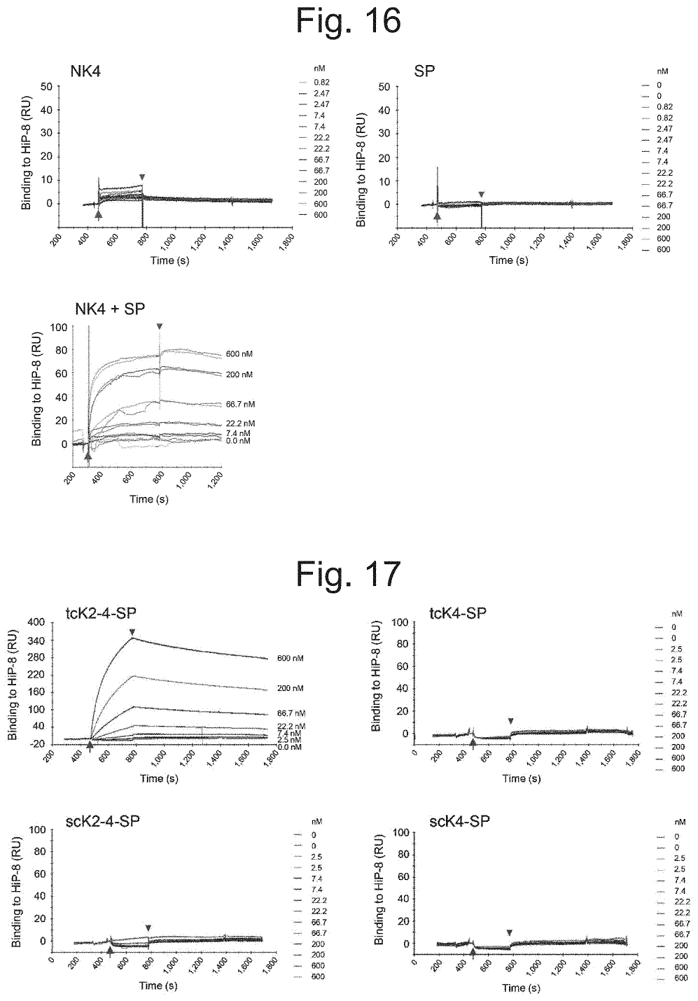

The results of the binding of HGF to HiP-8 are shown in FIG. 14. The results of the binding of HGF fragment ‘tcHGF (Xa)’ to HiP-8 and the binding of HGF fragment ‘scHGF(Xa)’ to HiP-8 are shown in FIG. 15. The results of HGF fragments, that is, NK4, SP, and a mixed system of NK4 and SP (NK4+SP) are shown in FIG. 16 and the results of the binding of tcKS-4-SP, tcK4-SP, scKS-4-SP and scK4-SP to HiP-8 are shown in FIG. 17.

Competition of HiP-8 against the binding between HGF or HGF fragment and MET ECD was measured by surface plasmon resonance (SPR) with Biacore 3000 (product of GE Flealthcare). His-labeled MET-ECD was supported on the surface of a nitrilotriacetic acid (NTA)-coated chip at 1000 RU. HiP-8 was mixed in advance at gradually increasing concentrations with 5 nM HGF, 30 nM NK4, or 30 nM SP fragment, and the resulting mixture was tested in 10 mM TBS (pH 7.4), 300 mM NaCl, and 0.05% Tween 20 (v/v) at a flow rate of 30 μl/min.

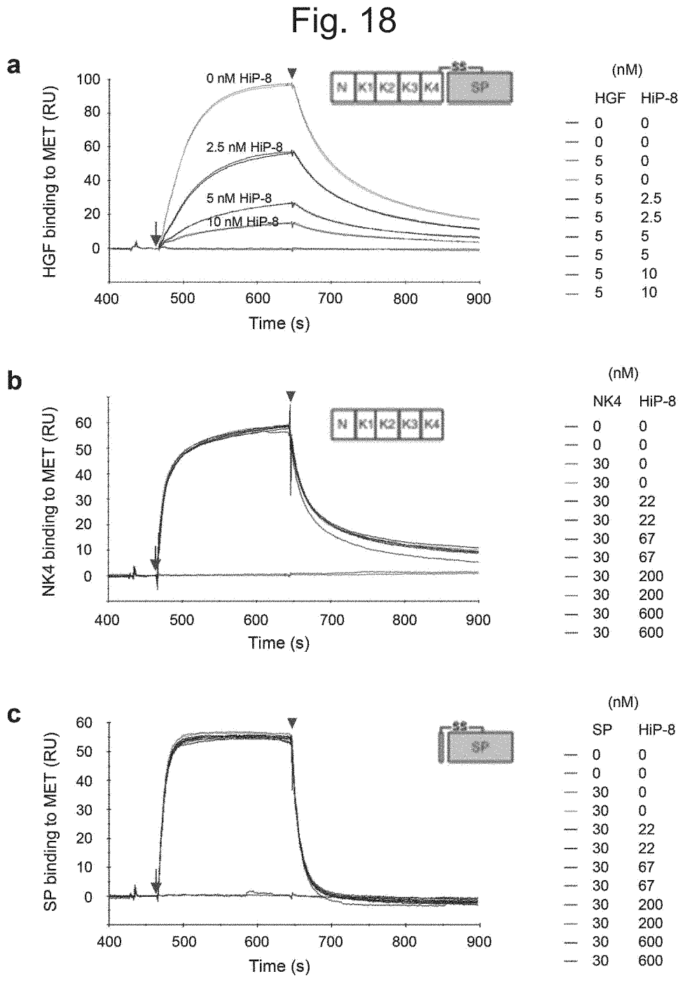

The competition of HiP-8 against the binding between HGF or HGF fragment and MET ECD is shown in FIG. 18.

(Change in Conformational Structure of HGF Caused by Cyclic Peptide)

System of HGF (2.5 μM) with or without HiP-8 were digested with trypsin at room temperature for 30 minutes. A trypsin:HGF molar ratio was set at 0:1, 0.004:1, 0.012:1, 0.037:1, 0.11:1, 0.33:1, and 1:1. The product obtained by digestion was analyzed on from 5 to 20% SDS-PAGE under reducing conditions and stained with Coomassie Blue.

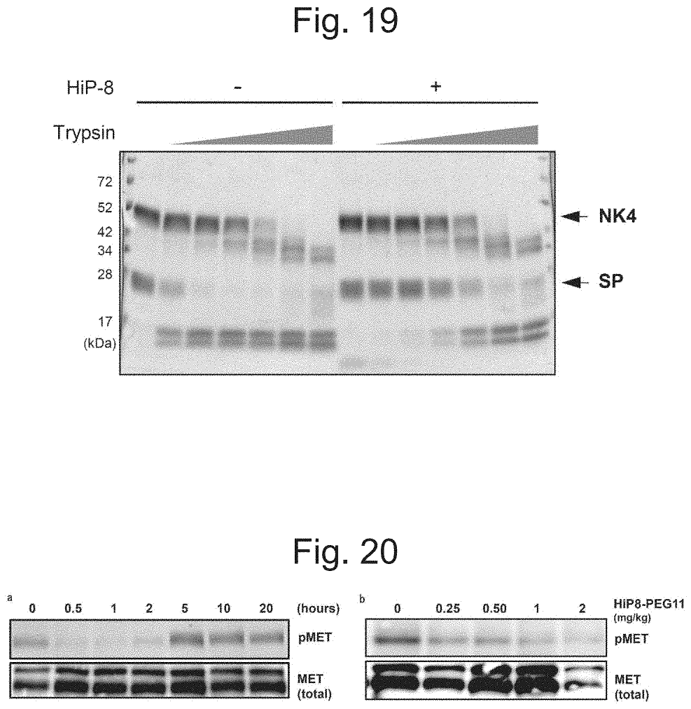

The results of electrophoresis are shown in FIG. 19. It has been found that HiP-8 induced a change in the conformation of HGF because no proteolysis at the SP chain portion of HGF was caused by trypsin.

(MET Activation Inhibition Test in Mice)

PC-9 human non-small cell lung cancer cells, which were to express human HGF, were transplanted subcutaneously to human HGF knocked-in SCID mice at 3×10⁶ cells/site. On day 28 after transplantation, HiP8-PEG11 in PBS/phosphate buffered physiological saline solution was intravenously injected. The tumor tissue was removed, and MET activated/tyrosine-phosphorylated and a total MET amount in the tumor tissue were quantitatively determined by western blotting with the tissue extract. It is to be noted that the tumor tissue was homogenized in a RIPA buffer solution. Each sample containing 10 mg of the protein was provided for SDS-PAGE and western blotting. Tyrosine-phosphorylated MET and a total MET amount were detected by western blotting.

The results of a time-dependent change (from 0 to 20 hours) of MET activated/tyrosine-phosphorylated (pMET) after HiP-8 administration and concentration-dependent inhibition by HiP8-PEG11 against MET activation/tyrosine phosphorylation are shown in FIG. 20.

(Tissue Staining)

A frozen human lung cancer tissue array FLU401B was obtained from US Biomax. A tissue section was fixed in a 4% (w/v) paraformaldehyde PBS solution at room temperature for 30 minutes, treated for one hour with a 3% (w/v) bovine serum-derived albumin (BSA) PBS solution to prevent non-specific adsorption of a probe, and incubated overnight at 4° C. with HiP-8-PEG11-biotin, a primary antibody against human HGF (clone: t5A11, Umitsu M, Sakai K, Ogasawara S, Kaneko M, Asaki R, Tamura-Kawakami K, Kato Y, Matsumoto K, Takagi J. Probing conformational and functional states of human hepatocyte growth factor by a panel of monoclonal antibodies. Scientific Reports, 6: 33149, 2016), or phosphorylated MET (phospho Y1230/1234/Y1235, product of Abeam) diluted with Can Get Signal (registered trademark) antibody staining (product of Toyobo).

After washing three times with PBS, the probes were detected with streptavidin or goat anti-mouse IgG labeled with Alexa Fluor 488 and goat anti-rabbit IgG (product of Thermo Fisher Scientific) labeled with Alexa Fluor 594.

Contrast staining of a cell nucleus was performed using 4′,6-diamidino-2-phenylindole (DAPI; product of Thermo Fisher Scientific). The tissue section was analyzed with a fluorescence microscope “Biozero BZ-9000 (KEYENCE). The results are shown in FIG. 21.

When in the human lung cancer tissue section, total HGF, that is, combination of scHGF and tcHGF was stained with a monoclonal antibody capable of recognizing both scHGF/single-chain HGF and tcHGF/two-chain HGF, total HGF was detected in many lung cancer cells (“scHGF+tcHGF”, the upper micrograph of the left column in FIG. 21). On the other hand, the tyrosine-phosphorylated MET, that is, pMET showing activated MET was detected partially in the lung cancer tissue. Overlapping region of total HGF and pMET was detected in some limited areas.

It has been confirmed, on the other hand, that when only tcHGF is detected using HiP-8, tcHGF positive region and pMET positive region overlap and agree well, and pMET (MET activation) depends on tcHGF.

HiP-8-PEG11 or HiP-8-PEG11-biotin was obtained by synthesizing as follows.

With regards to HiP-8-PEG11, solid-phase synthesis was started using, as a first amino acid, a Fmoc-protected PEG11 linker commercially available from Millipore followed by β-alanine, and then the core sequence of HiP-8 was synthesized. After chloroacetylation of the N terminal of HiP-8, a solution composed of 92.5% trifluoroacetic acid (TFA), 2.5% water, 2.5% triisopropylsilane, and 2.5% ethanedithiol was used to release the peptide from the resin (solid phase). The resulting peptide was precipitated by the addition of diethyl ether. For a cyclization reaction, the peptide pellets obtained by precipitation were dissolved in 10 mL of a dimethylsulfoxide (DMSO)/0.1% TFA aqueous solution (a 1:1 volume ratio) and the resulting solution was adjusted to pH>8 with triethylamine and incubated at 42° C. for one hour. The cyclization reaction was terminated by acidification with TFA. Then, the peptide was purified by reversed-phase high-performance chromatography (RP-HPLC, “Prominence LC-20AP”, product of Shimadzu) and Chromolith (registered trademark) prep column (200 mm×25 mm) of Merck, and the molecular weight was determined using a MALDI-TOF mass spectrum (Autoflex II, product of Bruker).

With regards to HiP-8-PEG11-biotin, monomethoxytritylated (MMT) Fmoc-Lys was used as an initiator amino acid, and synthesis of the core sequence of HiP-8 was completed by the same method as described above. Then, the monomethoxytrityl group on the Lys residue was selectively deprotected with a 1% TFA dichloromethane solution. The amine compound thus obtained was then reacted with 3 equivalents of D-biotin N-hydroxysuccinimide ester at room temperature for 3 hours. The peptide was cleaved from the solid phase and purified, and its molecular weight was determined by the above-described method.

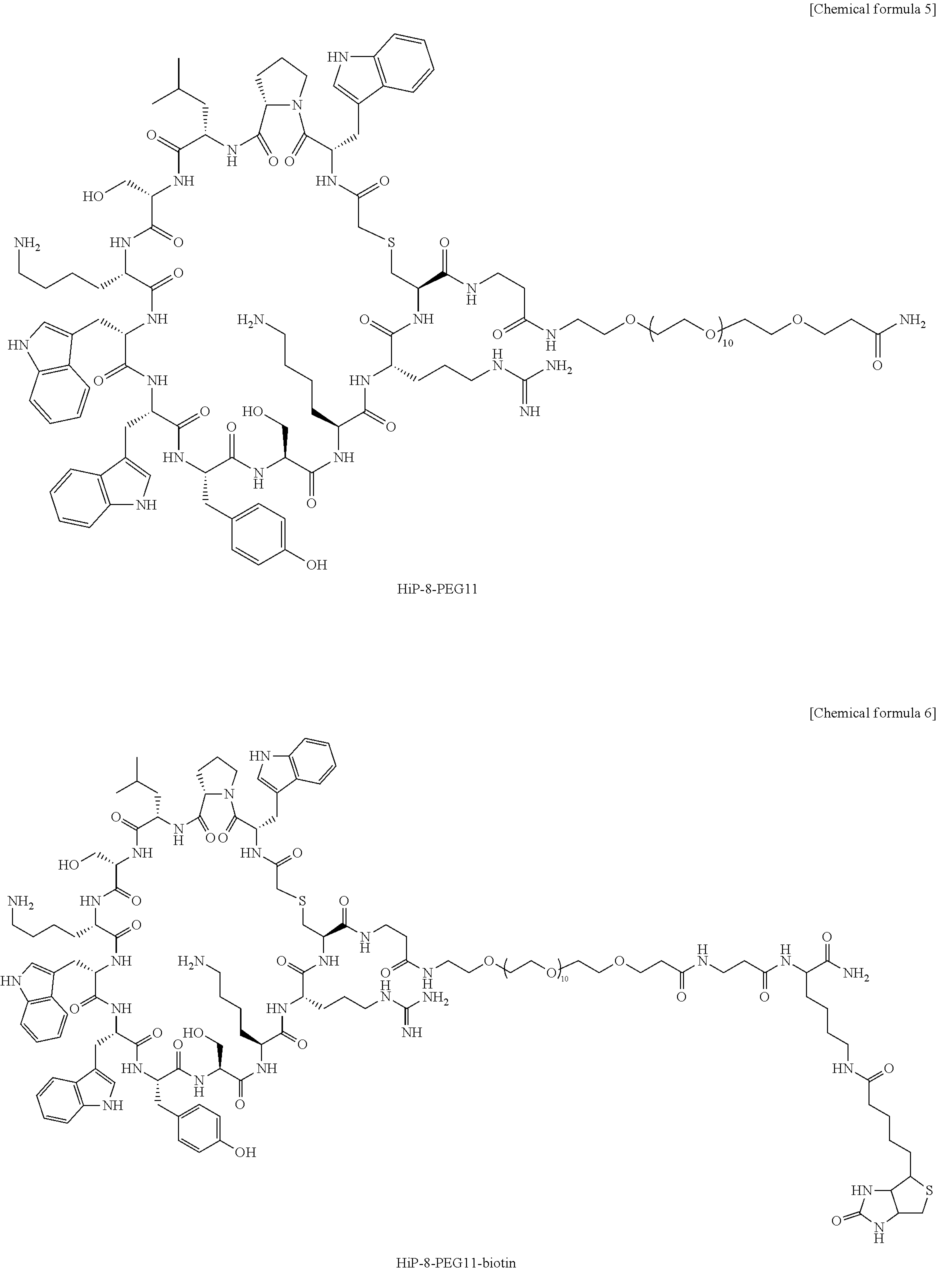

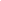

(Correlation Between Staining Intensity of tcHGF and Staining Intensity of pMET)

For semi-quantitative detection of antibody staining, stained region and mixed region in each tissue was quantitatively determined by ImageJ software (product of NIH).

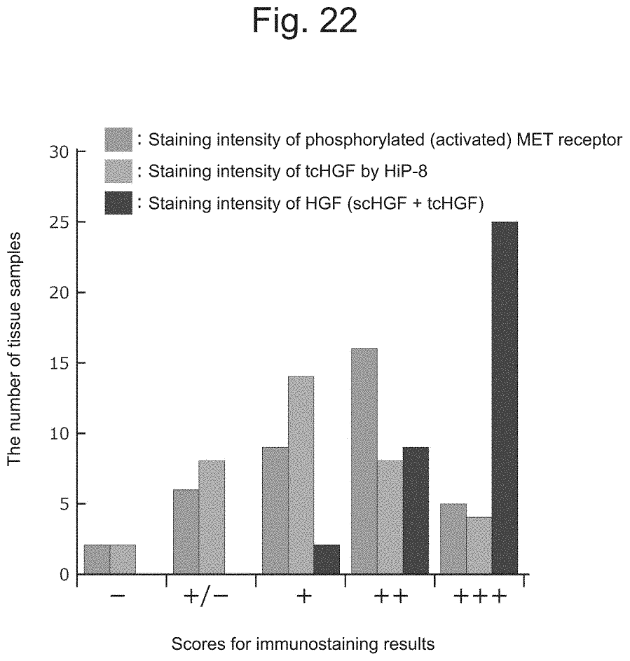

Samples were classified into − (no signal), + (very weak signals), + (weak signals), ++ (medium level of signals), and +++ (strong signals) based on the same standards. A graph of the intensities was made according to the above classification. The results are shown in FIG. 22.

The proportion of phosphorylated MET positive region or two-chain HGF positive region was calculated based on the same method as described above.

Thirty six samples of lung cancer tissue sections were each subjected to the same tissue staining as described in (tissue staining), and correlation in staining intensity among total HGF (scHGF+tcHGF), tcHGF (HiP-8), and pMET was studied. As a result, correlation was not found between total HGF and pMET, while the staining intensity of tcHGF detected by HiP-8 had strong correlation with the staining intensity of pMET.

1. A cyclic peptide having any unit structure selected from the structures represented by the following formula (1):
—X¹—X²—X³—X⁴—X⁵— (1)
(in the formula (1), X¹ is I, V or L, or an N-alkylamino acid thereof,
X² is S or T, or an N-alkylamino acid thereof,
X³ is K or an N-alkylamino acid thereof,
X⁴ is W or an N-alkylamino acid thereof, and
X⁵ is W, Y, H, or K, or an N-alkylamino acid thereof), or a pharmaceutically acceptable salt of the cyclic peptide.

2. The cyclic peptide or pharmaceutically acceptable salt thereof according to claim 1, wherein the X¹ is I or L, or an N-alkylamino acid thereof.

3. The cyclic peptide or pharmaceutically acceptable salt thereof according claim 1, wherein the number of amino acid residues constituting a cyclic structure is from 8 to 17.

4. The cyclic peptide or pharmaceutically acceptable salt thereof according to claim 1, wherein the cyclic peptide has an N—CO—CH₂—S structure.

5. The cyclic peptide or pharmaceutically acceptable salt thereof according to claim 4, wherein the N is derived from an amino group of tryptophan.

6. The cyclic peptide or pharmaceutically acceptable salt thereof according to claim 4, wherein the S is derived from a thiol group of cysteine.

7. A method of inhibiting hepatocyte growth factor (HGF) in a subject comprising administering the cyclic peptide or pharmaceutically acceptable salt thereof according to claim 1.

8. A pharmaceutical composition, comprising the cyclic peptide or pharmaceutically acceptable salt thereof according to claim 1.

9. The pharmaceutical composition according to claim 8, for use in the treatment or prevention of cancer-related diseases.

10. A method of detecting active MET and/or tcHGF, comprising using the cyclic peptide or pharmaceutically acceptable salt thereof according to claim 1.

11. A method of imaging a cancer tissue, comprising using positron emission tomography (PET) or chemiluminescence detection or fluorescence detection, or a combination thereof with the cyclic peptide or pharmaceutically acceptable salt thereof according to claim 1.

12. The cyclic peptide or pharmaceutically acceptable salt thereof according to claim 1, comprising a group for positron detection, a group for chemiluminescence detection and/or fluorescence detection, or a group for antibody staining detection.

13-15. (canceled)

16. A method of detecting active-MET and/or tcHGF in a subject, comprising:
bringing the cyclic peptide or pharmaceutically acceptable salt thereof according to claim 12 into contact with a tissue of the subject, followed by incubation; and
carrying out fluorescence detection, positron detection or antibody staining detection.

17. A positron emission tomography (PET) imaging method of a cancer tissue of a subject, comprising:
administering the cyclic peptide or pharmaceutically acceptable salt thereof according to claim 12 to the subject;
allowing the cyclic peptide or pharmaceutically acceptable salt thereof to penetrate the cancer tissue of the subject; and
acquiring a PET image of CNS or the cancer tissue of the subject.

18. A method for treating cancer, comprising administering the cyclic peptide or pharmaceutically acceptable salt thereof according to claim 1 to a cancer patient in need thereof.

19. A method of diagnosing cancer in a subject, comprising:
bringing the cyclic peptide or pharmaceutically acceptable salt thereof according to claim 12 into contact with a tissue of the subject, followed by incubation; and
carrying out fluorescence detection, positron detection or antibody staining detection.

20. A method of diagnosing cancer in a subject, comprising:
administering the cyclic peptide or pharmaceutically acceptable salt thereof according to claim 12 to the subject;
allowing the cyclic peptide or pharmaceutically acceptable salt thereof to penetrate the cancer tissue of the subject; and
acquiring a PET image of CNS or the cancer tissue of the subject.
